(19) United States
(12) Patent Application Publication
(10) Pub. No.: US 2024/0018269 A1
(43) Pub. Date: Jan. 18, 2024

(54) MODIFIED IMMUNOGLOBULIN WITH AFFINITY FOR FcGAMMARIIb AND METHOD OF USE THEREOF

(71) Applicants: MACFARLANE BURNET INSTITUTE FOR MEDICAL RESEARCH AND PUBLIC HEALTH LIMITED, Melbourne (AU); GENMAB A/S, Copenhagen (DK)

(72) Inventors: Phillip Mark HOGARTH, Williamstown (AU); Bruce David WINES, Heidelberg (AU); Halina Mary TRIST, Beaumaris (AU); Sandra Elizabeth ESPARON, Doncaster (AU); Alicia Michelle CHENOWETH, London (GB)

(21) Appl. No.: 18/213,344

(22) Filed: Jun. 23, 2023

Related U.S. Application Data
(63) Continuation of application No. PCT/AU2021/051548, filed on Dec. 22, 2021.

(30) Foreign Application Priority Data
Dec. 23, 2020 (AU) 2020904823

(51) Int. Cl. C07K 16/42; C07K 16/40; C07K 16/44

(52) U.S. Cl. CPC C07K 16/4291; C07K 16/40; C07K 16/44; C07K 2317/53; C07K 2317/72; C07K 2317/524; C07K 2317/76; C07K 2317/92

(57) ABSTRACT

Immunotherapeutic proteins comprising at least one heavy chain polypeptide derived from an IgG2 antibody are disclosed, wherein the heavy chain polypeptide comprises at least constant heavy domains 2 and 3 (CH2 and CH3) and the lower hinge, and the sequence of the lower hinge comprises a mutation enabling the immunotherapeutic protein to bind to and/or activate FcγRIIb. The immunotherapeutic protein is suitable for use in methods of treating diseases or conditions wherein, for example, the activation of FcγRIIb (ie for recruitment of the inhibitory functions of FcγRIIb) is beneficial, such as allergic diseases.

### CROSS-REFERENCE TO RELATED APPLICATIONS

The present application is a continuation application of International Patent Application No. PCT/AU2021/051548 filed Dec. 22, 2021, which application claims priority from Australian Patent Application No. 2020904823 titled “Modified immunoglobulin and method of use thereof (1)” and filed on 23 Dec. 2020, the content of which is hereby incorporated by reference in its entirety.

### SEQUENCE LISTING

The instant application contains a Sequence Listing which has been submitted electronically in XML format and is hereby incorporated by reference in its entirety. Said XML copy, created on Oct. 11, 2023, is named 137412-0200_SL.xml and is 47,642 bytes in size.

### TECHNICAL FIELD

The present disclosure relates to immunotherapeutic proteins comprising modified immunoglobulin molecules (ie antibodies) for use in methods of treating diseases or conditions. More particularly, the present disclosure describes mutant IgG2 antibodies showing improved binding specificity and affinity to the human inhibitory receptor, FcγRIIb. These antibodies may be used for treating diseases wherein, for example, the activation of FcγRIIb is beneficial, such as allergic diseases.

### BACKGROUND

Monoclonal antibodies (mAbs) have become one of the most important and successful types of therapeutics, revolutionising the treatment of cancer and inflammatory diseases such as autoimmune diseases. Many mAbs engineered on an IgG antibody class backbone specifically harness the powerful effector functions of the immune system by engaging both the target antigen via their variable Fab domains and Fcγ receptors (FcγRs) via their heavy chains including the constant Fc fragment. In inflammatory diseases, engineered mAbs may potentially act by neutralising inflammatory mediators, by neutralising their receptors or by engaging immune regulatory receptors. One particular interest in the context of the present invention, is the potential to neutralise the pro-inflammatory responses in allergic reactions, the major mediator of which is allergen-specific IgE activation of its high affinity receptor, FcεRI.

An example of an engineered mAb approved for use in the treatment of allergic diseases is the IgG1-based mAb, Omalizumab. This mAb targets the IgE/FcεRI pathway by neutralising the interaction of IgE with FcεRI to thereby prevent activation of basophils, which are a critical inflammatory cell in IgE-dependent allergic responses (Gericke J et al., JEADV 29(9):1832-1836, 2014). Human basophils, express both FcεRI, as well as its regulator, the inhibitory receptor FcγRIIb (Kepley C L et al, J Allergy Clin Immunol 106(2):337-348, 2000). FcγRIIb is a potent checkpoint regulating antibody-dependent inflammatory cell activation. It acts via an Immunoreceptor Tyrosine Inhibitory Motif (ITIM) that modulates the Immunoreceptor Tyrosine Activation Motif (ITAM)-dependent signalling pathway of FcεRI and the activating-type IgG and IgA Fc receptors, namely FcγRI, FcγRIIa, FcγRIIIa and FcαRI. FcγRIIb also modulates B cell activation by the B cell antigen receptor (BCR). Targeting of immune checkpoints such as FcγRIIb has emerged as a strategy for modulating leukocyte responses in disease (Kaplon H and J M Reichert, Mabs 11(2):219-238, 2019; Chenoweth A M et al., Immunol Cell Biol 98(4):287-304, 2020). However, unlike mAb targeting of checkpoints of T cell function (eg PD-1, PD-L1), FcγRIIb is unique in the context of mAb therapeutics, because of its specificity for the Fc fragment of the therapeutic mAb, and since, to exert its inhibitory action, FcγRIIb requires co-crosslinking with an ITAM-containing activating receptor, for example an activating type FcR complex or the antigen receptor complex of B cells also known as the B cell antigen receptor or BCR (Getahun A and J C Cambier, Immunol Reviews 268(1):66-73, 2015; Chenoweth A M et al., 2020 supra), a strategy which uses specific immune-suppressing antibodies that can harness the normal physiological inhibitory role of FcγRIIb (whilst co-aggregated with FcεRI or co-aggregated with the B-cell antigen receptor, BCR) in an allergy response, offers a potentially useful way to target activating receptors such as the high affinity IgE receptor, FcεRI, or the BCR.

In work leading to the present disclosure, the inventors chose to investigate the use of IgG2-based immunoglobulin molecules for the treatment or prevention of an allergic response, since the “IgG2 backbone” is considered to be “functionally inert” inasmuch as IgG2 antibodies have a very restricted FcγR specificity binding only to one allelic form of the low affinity activating-type FcγRIIa (ie the “His 131 form”, FcγRIIa-His131; Bredius R G M et al., J Immunol 151:1463-1472, 1993), limited effector function and are unable to fix complement; meaning that an engineered IgG2 antibody (ie a mutant IgG2 antibody) may be less prone to causing unwanted activities (ie “side effects”) including, unwanted pro-inflammatory effector responses such as FcγR-dependent cytokine release from an inflammatory cell, cell cytotoxicity from a killer cell or, potentially, life-threatening inflammatory responses known as a “cytokine storm” (eg as has been observed with TGN1412, an IgG4-based anti-CD28 monoclonal antibody; Suntharalingam G et al., N Engl J Med 355(10):1018-1028, 2006). However, while IgG2 antibodies are known not to bind to FcγRIIb or the other FcγRs, with the exception of the one allelic form of FcγRIIa mentioned above (Bruhns P et al, Blood 113(16):3716-3725, 2009; and FIG. 1A hereinafter), the inventors were nevertheless, and surprisingly, able to engineer the IgG2 backbone so as to improve FcγRIIb specificity, affinity and inhibitory potency. It is considered that the approach taken has wider implications for the generation of immunotherapeutic proteins including novel potent anti-inflammatory therapeutic mAbs and molecules.

### SUMMARY

Thus, in a first aspect, the present disclosure provides a method of treating a disease or condition in a subject, wherein binding to and/or activation of FcγRIIb is beneficial in the treatment or prevention of said disease or condition, said method comprising administering to said subject an effective amount of an immunotherapeutic protein comprising at least one heavy chain polypeptide derived from an IgG2 antibody, wherein said heavy chain polypeptide comprises at least constant heavy domains 2 and 3 (ie CH2 and CH3) and the lower hinge, and the sequence of the lower hinge comprises a mutation enabling the immunotherapeutic protein to bind to and/or activate FcγRIIb.

In some particular preferred embodiments, the lower hinge sequence of the immunotherapeutic protein comprises an amino acid sequence selected from: ELLGG (SEQ ID NO: 1), EFLGG (SEQ ID NO: 2) and EFEGG (SEQ ID NO: 3).

In a second aspect, the present disclosure provides the use of an immunotherapeutic protein as defined in the first aspect, for treating a disease or condition wherein binding to and/or activation of FcγRIIb is beneficial, including, for example, allergic diseases, autoimmune diseases and conditions, infectious diseases and proliferative diseases.

In a third aspect, the present disclosure provides the use of an immunotherapeutic protein as defined in the first aspect, in the manufacture of a medicament for treating a disease or condition wherein binding to and/or activation of FcγRIIb is beneficial, including, for example, allergic diseases, autoimmune diseases and conditions, other inflammatory diseases, infectious diseases and proliferative diseases.

In a fourth aspect, the present disclosure provides a pharmaceutical composition or medicament comprising an immunotherapeutic protein as defined in the first aspect, and a pharmaceutically acceptable carrier, diluent and/or excipient.

### BRIEF DESCRIPTION OF FIGURES

FIG. 1 shows the binding profiles of mutant IgG2 antibodies according to the present disclosure with human FcγR in comparison with IgG1, IgG2, IgG4 and mutant IgG4 antibodies;

FIG. 2 shows inhibition of bee venom Api m 1 allergen-dependent basophil activation in washed blood, that lacks physiological levels of IgG, from allergic patients using mutant IgG2 antibodies according to the present disclosure;

FIG. 3 provides the results of basophil activation test (BAT) assays using mutant IgG2 antibodies according to the present disclosure, showing inhibition of bee venom Api m 1 allergen-dependent basophil activation in whole blood, that contains physiological levels of IgG, from allergic patients;

FIG. 4 provides the results of assays conducted to determine whether mutant IgG2 antibodies including SELF mutations show improved binding affinity to FcγRIIb and FcγRIIa-R131: (A) BLI analysis of the binding of rsFcγRIIb to monomeric anti-TNP IgG1, IgG2 and the indicated mutants or IgG2 captured on TNP-BSA (mean±SEM); (B) BLI analysis of the binding of rsFcγRIIIa-R131 to monomeric anti-TNP IgG1-SELF, IgG2-SELF, IgG2-FLGG-SELF and IgG2-FEGG-SELF captured on TNP-BSA (mean±SD);

FIG. 5 provides results demonstrating that mutant IgG2 antibodies according to the present disclosure have altered human FcγR binding profiles. Using cells expressing human FcγR, the binding avidity of complexed IgG to the low affinity human FcγR or affinity of un-complexed monomeric IgG to FcγRI was determined by flow cytometry: (A) FcγRIIb, (B) FcγRIIa-R131, (C) FcγRIIa-H131, (D) FcγRIIIa-F158, (E) FcγRIIIa-V158 and (F) FcγRI. Statistical comparisons were made between IgG mutants and the relevant IgG WT backbone. *(p<0.5), **(p<0.1), ***(p<0.01), ****(p<0.0001), n.s (not significant);

FIG. 6 shows the results of FcγR binding specificity of monomers of mutant IgG2 antibodies according to the present disclosure. Some of the mutant IgG2 antibodies included the SELF mutations;

FIG. 7 provides the results of BAT assays using mutant IgG2 antibodies according to the present disclosure (some with the SELF mutations), showing inhibition of anti-IgE dependent basophil activation by IgG mutants (using anti-IgE-TNP as IgE-dependent stimuli) in whole blood from healthy donors; and

FIG. 8 provides the results of experiments to assess FcγRIIb expression and FcγRIIb specific inhibition of FcεRI activation: (A) Flow cytometric detection of FcγRIIb on IgE positive basophils using F(ab′)2 fragments of the FcγRIIb specific mAb, H2B6, compared to buffer background (Bkg); (B) H2B6 F(ab′)2 blockade of FcγRIIb prevents inhibition of basophil activation by IgG2-FLGG (7.5 μg/ml) in whole blood BAT; (C) mAb suppression of IgE/FcεRI-dependent induced calcium mobilisation in IIA1.6 cells co-expressing the FcεRI (αβγ) complex and inhibitory FcγRIIb. The cells were pre-treated with IgE overnight and stimulated with anti-TNP antibodies pre-complexed with TNP-conjugated F(ab′)2 anti hIgE. Cells were stimulated with anti-IgE-TNP (F(ab′)2) (20 μg/ml) and mAbs (35 μg/ml). Calcium Flux (340/380 nm) was measured over time and inhibition of calcium flux by mutant IgG2 antibodies compared to parental wild type IgG2 which does not bind FcγRIIb. Unstimulated baseline control is buffer alone (n=3);

FIG. 9 provides the results of experiments showing suppression of antigen stimulation of B cells by IgG mAbs detecting the B cell antigen receptor complex. Anti-IgE mAbs with the variable domains of omalizumab with an IgG4 backbone or IgG2 backbone suppress NIP(22)BSA stimulation of the NIP-specific hu-IgE BCR-triggered calcium flux in B cells co-expressing human FcγRIIb1. Regulation of the IgE BCR had the hierarchy of IgG1 (omalizumab)˜IgG4>IgG2, which correlated with the rank of FcγRIIb binding activity of these IgG formats; and

FIG. 10 provides the results of experiments showing suppression of antigen stimulation of B cells by IgG2 mutant mAbs (according to the present disclosure) binding to the B cell antigen receptor complex. The anti-IgE mAbs with the variable domains of omalizumab provided as an IgG2 mutant antibody according to the present disclosure suppress antigen (ie NIP(22)BSA) stimulation of the NP-specific hu-IgE BCR-triggered calcium flux in B cells co-expressing human FcγRIIb1. The effect of the mutations on regulation of the IgE BCR by the IgG2 mutant mAbs had the hierarchy of FLGG-SELF>FLGG˜FEGG-SELF>FEGG>IgG2, which broadly correlated with the rank of FcγRIIb binding activity of these mutations in the IgG2 format.

### DETAILED DESCRIPTION

The inventors have found that the functionally inert IgG2 backbone may be used as a scaffold to incorporate mutation(s) of the lower hinge sequence (eg to effectively replace the lower hinge with the lower hinge sequence of IgG4) and other mutations to improve binding specificity and affinity to the human inhibitory receptor, FcγRIIb (including any or all of the mRNA splice variants well known to those skilled in the art, that is FcγRIIb1, FcγRIIb2 and FcγRIIb3 (Getahun A and J C Cambier, 2015 supra; and Chenoweth A M et al., 2020 supra; Anania J C et al., Front Immunol 9:1809, 2018). The resulting mutant IgG2 antibodies can, for example, potentially provide enhanced agonistic function of mAbs where FcγR “scaffolding” is required for therapeutic effect. They can also be useful for the development of non-agonistic therapeutic antibodies in circumstances where harnessing the normal physiological inhibitory function of FcγRIIb, or antigen or immune complex clearance mediated by FcγRIIb (eg by endocytosis/internalisation or “sweeping”), is desirable.

Thus, in a first aspect, the present disclosure provides a method of treating a disease or condition in a subject, wherein binding to and/or activation of FcγRIIb is beneficial in the treatment or prevention of said disease or condition, said method comprising administering to said subject an effective amount of an immunotherapeutic protein comprising at least one heavy chain polypeptide derived from an IgG2 antibody, wherein said heavy chain polypeptide comprises at least constant heavy domains 2 and 3 (ie CH2 and CH3) and the lower hinge, and the sequence of the lower hinge comprises a mutation enabling the immunotherapeutic protein to bind to and/or activate FcγRIIb.

The at least one heavy chain polypeptide of the immunotherapeutic protein may be any heavy chain polypeptide that those skilled in the art will recognise as being derived from an IgG2 antibody, for example for the reason that the CH2 and CH3 domains of the heavy chain polypeptide comprise an amino acid sequence that shows at least 95%, preferably at least 98%, identity to the sequences of the CH2/CH3 domains of wild type (WT) IgG2 antibodies or, more preferably, to the sequences of the CH2/CH3 domains of WT human IgG2 antibodies such as provided by Wines B D et al., J Immunol 197(4):1507-1516, 2016 and GenBank Accession No: AH005273.2).

Those skilled in the art will readily understand that the heavy chain polypeptide derived from an IgG2 antibody may, for example, comprise a full length heavy chain polypeptide (ie comprising the constant heavy region (CH) and the variable heavy (VH) region), or it may comprise a fragment thereof which comprises at least CH2, CH3 and the lower hinge. One preferred example of such a fragment is the Fc fragment which corresponds to one of the heavy chain components of the fragment generated by papain digestion of an antibody (cleaving the polypeptides within the upper hinge sequence to generate an Fc fragment comprising two heavy chain cross-linked fragments, each comprising CH2, CH3 and the lower and core hinge sequences). Similar heavy chain polypeptides may be prepared through digestion of an antibody with plasmin and human neutrophil elastase (NHE), also known to those skilled in the art as generating “Fc fragments”, and such heavy chain polypeptides may suitably comprise the immunotherapeutic protein of the method of the first aspect. Further examples of suitable heavy chain polypeptides may comprise, in addition to CH2, CH3 and the lower hinge, all or part of the constant heavy domain 1 (CH1) of an IgG2 antibody, and/or the core hinge and/or upper hinge sequences.

In some embodiments, the immunotherapeutic protein may comprise a heavy chain polypeptide provided in the form of a fusion protein or protein conjugate. Those skilled in the art will understand that in a fusion protein, the heavy chain polypeptide will be covalently linked (ie “fused”) to a polypeptide or peptide partner (ie a fusion partner) via a peptide bond or short peptide linker sequence at the N- or C-terminus of the fusion partner, whereas in a protein conjugate, it is to be understood that the heavy chain polypeptide will be covalently or non-covalently linked to a conjugate partner (which may be a polypeptide or peptide, or other chemical entity) through a chemical linkage such as a disulphide bond or crosslinker compound such as a homobifunctional crosslinker such as disuccinimidyl suberate (DSS) (eg bis(sulfosuccinimidyl)suberate (BS3); ThermoFisher Scientific, Waltham, MA, United States of America) or disuccinimidyl tartrate (DST) to link amine groups or a heterobifunctional crosslinker such as m-maleimidobenzoyl-N-hydroxysuccinimide ester (MDS) and N-(ε-maleimidocaproloxy) succinimide ester (EMCS), or by other non-covalent bonding such as hydrogen bonding. Where the conjugate partner is a polypeptide, the protein conjugate may otherwise be considered as a cross-linked protein. The conjugate partner may be conjugated to the heavy chain polypeptide at the N- or C-terminus, but otherwise may be conjugated at any other suitable site on the heavy chain polypeptide (eg within CH1 or the upper hinge sequence if these are included in the heavy chain polypeptide). Those skilled in the art will recognise that the fusion partner or conjugate partner may provide one or more useful activity or function. For example, the fusion partner may improve protein recovery or expression (eg Human serum albumin (HSA), and Glutathione S-transferase (GST)), provide various affinity-tags such as a polyhistidine tag (His-tag) or a FLAG-tag, or an additional ability to bind to an antigen of interest. Other examples of a fusion partner or conjugate partner include receptors (eg a cytokine receptor such as a receptor for an interleukin (eg IL-1 receptor) or a receptor for a cytokine of the TGF-0 superfamily), or cell surface molecules and immune checkpoints (eg CTLA4 or PD1).

In some other embodiments, the immunotherapeutic protein may comprise at least one heavy chain polypeptide that is provided in a dimeric or multimeric form. For example, the heavy chain polypeptide may have a natural propensity to form covalently linked dimers through one or more cysteine (C) residue, particularly where situated within the hinge sequence, especially the core hinge sequence (Yoo E M et al., J Immunol 170:3134-3138, 2003). Techniques suitable for producing multimeric forms of the heavy chain polypeptide (eg with 3, 4, 5, 6 etc. copies of the heavy chain polypeptide) have been described and are well known to those skilled in the art (eg Fc multimeric forms (Stradomers™) comprising linked multimerisation domain (MD) sequences from the hinge region of human IgG2 or the isoleucine zipper (ILZ) to the N- or C-terminus of murine IgG2a; Fitzpatrick E A et al., Front Immunol 11, article 496, 2020). Such dimeric or multimeric forms of the heavy chain polypeptide are preferably soluble (ie in physiological saline).

In still some other embodiments, the immunotherapeutic protein comprises an IgG2 antibody comprising two full length heavy chain polypeptides with two light chain polypeptides, particularly a mutant IgG2 antibody wherein at least one of the two heavy chain polypeptides comprises a mutation in the lower hinge sequence enabling the IgG2 antibody to bind to and/or activate FcγRIIb.

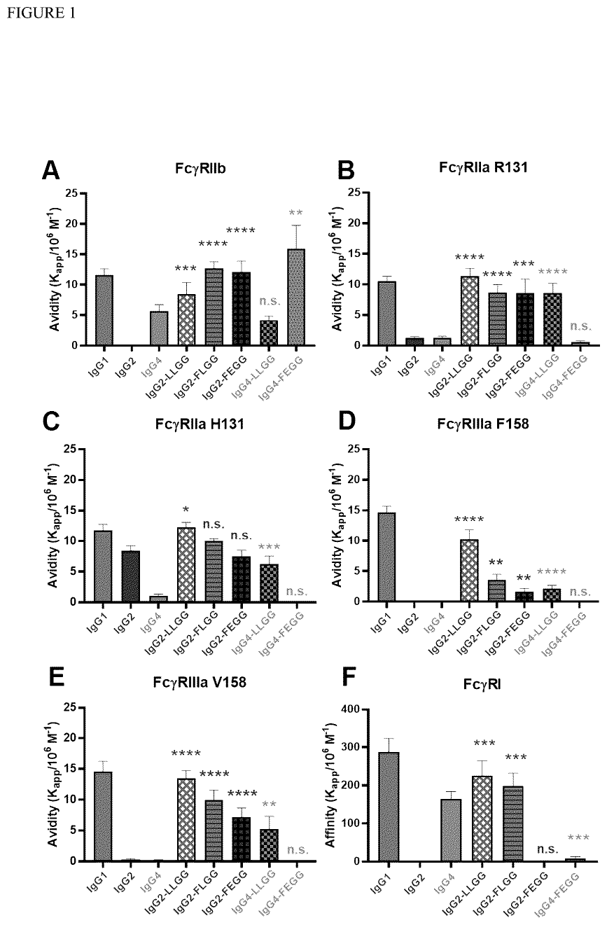

Wild type (WT) human IgG2 antibodies show no, or virtually undetectable, binding to FcγRIIb (see FIG. 1A). In contrast, the method of the present disclosure may utilise a mutant human IgG2 antibody (ie an IgG2 antibody comprising a mutation in a lower hinge sequence of at least one heavy chain polypeptide) which enables the IgG2 antibody to bind to and/or activate FcγRIIb. The mutation in the IgG2 lower hinge sequence may comprise the substitution of the sequence, or the substitution of one or more amino acid(s) within the sequence, at positions 233-236 (EU numbering system; Edelman G M et al., Proc Natl Acad Sci USA 63:78-85, 1969, and Kabat E A, Sequences of Proteins of Immunological Interest, 5th ed., DIANE Publishing, PA, USA, 1991; but for the avoidance of any doubt, the IgG2 lower hinge sequence as referred to herein comprises the amino acids at the positions equivalent to those of the lower hinge sequence of IgG1), which is PVAG (SEQ ID NO: 14) in human IgG2 antibodies. The mutation in the IgG2 lower hinge sequence may further comprise an amino acid insertion or addition; for example, a mutated human IgG2 lower hinge (PVAG: SEQ ID NO: 14) sequence may be a five (5) amino acid sequence in the mutant IgG2 antibody.

Preferably, in the immunotherapeutic protein, the lower hinge sequence comprises the amino acid sequence:

X1X2X3-G-X5 (SEQ ID NO: 4)
wherein
X1 is selected from proline (P) and glutamic acid (E),
X2 is selected from valine (V), leucine (L) and phenylalanine (F),
X3 is selected from leucine (L), alanine (A) and glutamic acid (E), and
X5 is selected from glycine (G) and proline (P), or is absent (ie such that the sequence is X1X2X3-G: SEQ ID NO: 31),
but with the proviso that the lower hinge does not consist of a wild type IgG2 lower hinge sequence (eg PVAG (SEQ ID NO: 14) of human IgG2 antibodies).

In some particular preferred embodiments, the lower hinge sequence of the mutant IgG2 antibody comprises an amino acid sequence selected from: ELLGG (derived from human IgG1; SEQ ID NO: 1), EFLGG (derived from human IgG4; SEQ ID NO: 2), EFLGP (SEQ ID NO: 5) and EFEGG (SEQ ID NO: 3).

As mentioned above, the immunotherapeutic protein of the present disclosure (eg a mutant IgG2 antibody) may bind to and/or activate FcγRIIb. Where the immunotherapeutic protein binds to and activates FcγRIIb, the immunotherapeutic protein may act so as to elicit FcγRIIb inhibitory function. As such, in some embodiments, the method of the present disclosure is particularly suited to the treatment or prevention of diseases or conditions wherein the inhibitory effects of FcγRIIb are beneficial. Accordingly, in some preferred embodiments, the method of the present disclosure is particularly directed to the treatment or prevention of, for example, an allergic disease, wherein the binding and activation of FcγRIIb by the immunotherapeutic protein mediates FcγRIIb-dependent inhibition of allergic basophil activation by IgE.

Wherein the method of the present disclosure is conducted for the treatment or prevention of an allergic response, the immunotherapeutic protein is preferably one that comprises the mutant lower hinge sequence EFLGG (SEQ ID NO: 2) or ELLGG (SEQ ID NO: 1), since in the example described hereinafter, it was found that the mutant IgG2 antibodies denoted as the “IgG2-FLGG (SEQ ID NO: 6)” mAb and the “IgG2-LLGG (SEQ ID NO: 7)” mAb were the most potent inhibitors of IgE/FcεRI basophil activation of the mutant IgG2 antibodies tested, despite a relatively low affinity for FcγRIIb (nb. they only bound as an immune complex).

While in some preferred embodiments the immunotherapeutic protein includes no further mutation(s) within the heavy chain polypeptides (or, at least, within the constant heavy region of the heavy chain polypeptides), in some other embodiments, it may be advantageous to further include one or more additional mutation(s) of at least one heavy chain polypeptide. For example, the immunotherapeutic protein (eg a mutant IgG2 antibody) may further comprise an amino acid substitution(s) at position 267 and/or 328 (EU numbering) in the CH2 domain of at least one, and more preferably both, of the heavy chain polypeptides, such as the so-called “SELF” mutations, S267E and L328F substitution(s) respectively. In the example described hereinafter, it was found that a mutant IgG2 antibody with an EFEGG (SEQ ID NO: 3) mutant lower hinge sequence and the SELF mutations (eg “IgG2-FEGG (SEQ ID NO: 8)-SELF” mAb) bound to FcγRIIb in the most specific manner of the mutant IgG2 antibodies tested and retained inhibitory potency.

However, while the SELF mutations enhanced the interaction of the mutant IgG2 antibodies with FcγRIIb, in vivo the specificity of such mutant IgG2 antibodies in a subject may be determined by the presence of the high/low responder polymorphism of FcγRIIa, since it has been found that the antibodies including the SELF mutations have high affinity interactions with FcγRIIb and the activating receptor-type FcγRIIa, but only the “Arg 131 form”, not the “His 131 form”. Accordingly, in some embodiments, where the method involves the administration of a mutant IgG2 antibody including the SELF mutations, the method may preferably be intended for use with a subject that is homozygous for FcγRIIa-H131 (nb. subjects that are homozygous for FcγRIIa-H131 represent about 30% of the population; van der Pol W L and J van de Winkel, Immunogenetics 48:222-232, 1998). In such embodiments, the method may further comprise a step of selecting the subject by genotyping for the high/low responder polymorphism of FcγRIIa. That is, in some embodiments, a subject determined to be homozygous for FcγRIIa-H131 may be selected for treatment by administering a mutant IgG2 antibody comprising the SELF mutations.

Where the immunotherapeutic protein comprises a mutant IgG2 antibody, the mutant IgG2 antibody will typically comprise a monoclonal antibody (mAb) and is preferably a human mAb or humanised mAb. Such antibodies may be produced in accordance with any of the standard methodologies known to those skilled in the art. For instance, those skilled in the art can readily prepare a mutant IgG2 antibody suitable for use in the method of the present disclosure by generating a construct(s), using standard molecular biology techniques, which comprises a polynucleotide sequence(s) encoding the variable heavy (VH) and light (VL) region sequence of a suitable antibody (eg one including an antigen binding region that binds to an antigen of interest) and a constant heavy (CH) region from an IgG2 antibody (eg as previously described in Wines B D et al., 2016 supra), and incorporate into the CH region-encoding polynucleotide sequence by standard molecular biology techniques such as site-directed mutagenesis, polynucleotide sequence changes to encode the lower hinge sequence mutations described above (and SELF mutations where desired). The construct(s) can be introduced into a suitable host cell (eg a human kidney (HEK) host cell), cultured according to standard culturing protocols and the expressed mutant IgG2 antibody purified from the culture supernatant using, for example, any of the known suitable methodologies for purification (eg affinity chromatography).

Treatment of Diseases Through Activation of FcγRIIb

In some embodiments, the method of the present disclosure may be used for treating a disease or condition in a subject, wherein activation of FcγRIIb (ie for recruitment of inhibitory action) is beneficial in the treatment or prevention of said disease or condition.

In some examples of such embodiments, the immunotherapeutic protein may target an ITAM signalling receptor complex by, for example, including a binding domain such as an antigen binding region (eg an Fab region) that recognises a component of a potential signalling complex such as, for example, (a) an antigen (eg an allergen or autoantigen bound to an antibody such as an IgG, IgE or IgA which is bound to a receptor); (b) an antibody bound to an activating receptor; (c) an antibody (ligand) binding domain of an activating receptor; or (d) a subunit of an activating receptor (eg the Fc receptor common γ chain), while in other examples, the immunotherapeutic protein may target a component of the B cell antigen receptor (BCR) complex that includes, for example, (a) an antigen bound to an immunoglobulin component of a BCR complex (eg an allergen, autoantigen, an antigen of an infectious agent such as an antigen of a bacterial or viral pathogen, or an antigen from a transplanted tissue or organ), or (b) a subunit of a BCR complex (eg a membrane immunoglobulin of a BCR complex such as an IgM, IgD, IgG, IgE or IgA) or an associated Ig-α or β chains (eg CD79a or CD79b; or CD19, CD21 or CD81).

In all of such examples, the immunotherapeutic protein will bring about the necessary co-cross-linking of an ITAM-containing activating receptor with the inhibitory receptor, FcγRIIb, to recruit the inhibitory action of FcγRIIb and, as will be appreciated from the above discussion, this may be achieved in a number of different ways wherein the target of the immunotherapeutic protein varies considerably and thus enables their potential use for the treatment or prevention of a wide range of different diseases or conditions.

More particularly, in some examples where the immunotherapeutic protein is intended to be used for the treatment or prevention of a disease or condition by binding to and activating FcγRIIb, the immunotherapeutic protein may comprise a binding domain such as an antigen binding region targeted to an antigen of interest present in an immune complex (ie where the antigen is complexed with IgE, IgG or IgA) bound via the Fc portion of the immunoglobulin to FcεRI, or an activating type FcγR (eg FcγRI, FcγRIIa, FcγRIIc, and FcγRIIIa or FcγRIIIb; otherwise known as CD64, CD32a, CD32c, CD16a and CD16b respectively) or the activating type receptor, FcαRI (CD89). As such, the antigen of interest may be selected from, for example: allergens (eg bee venom) for the use of the immunotherapeutic protein for treatment or prevention of allergic diseases; autoantigens for the use of the immunotherapeutic protein for treatment or prevention of autoimmune diseases (eg autoantigens associated with systemic lupus erythematosus (SLE) or multiple sclerosis (MS)); antigens associated with other inflammatory diseases such as immune complex vasculitis, antigens from a transplanted tissue or organ to enable use of the immunotherapeutic protein for treatment or prevention of antibody-mediated transplant rejection; and antigens of infectious agents such as an antigen of a bacterial or viral pathogen (eg an antigen of the SARS-CoV-2 virus or dengue virus).

Alternatively, in some examples where the immunotherapeutic protein is intended to be used for the treatment or prevention of a disease or condition by binding to and activating FcγRIIb, the immunotherapeutic protein may comprise a binding domain such as an antigen binding region targeted to an immunoglobulin present in an immune complex bound via the Fc portion of the immunoglobulin to FcεRI, or an activating type FcγR (eg FcγRI, FcγRIIa, FcγRIIc, and FcγRIIIa or FcγRIIIb) or the activating type receptor, FcαRI. That is, where an antigen is complexed with IgE, IgG or IgA, the immunotherapeutic protein may be targeted to the heavy chain or light chain of IgE, IgG or IgA to bring about the co-cross-linking of FcγRIIb and an activating Fc receptor. As such, an immunotherapeutic protein targeted in this way can also be used for the treatment or prevention of allergic diseases (eg where an allergen is complexed with the targeted immunoglobulin), autoimmune diseases such as SLE and MS (eg where the autoantigen is complexed with the immunoglobulin targeted by the immunotherapeutic protein), other inflammatory diseases such as immune complex vasculitis (eg where the relevant antigen is complexed with the targeted immunoglobulin), antibody-mediated transplant rejection (eg where the antigen from a transplanted tissue or organ is complexed with the targeted immunoglobulin), and infectious diseases (eg where the antigen of an infectious agent is complexed with the targeted immunoglobulin), but also proliferative diseases (eg where a cancer antigen is complexed with the immunoglobulin targeted by the therapeutic protein).

Further, in some examples where the immunotherapeutic protein is intended to be used for the treatment or prevention of a disease or condition by binding to and activating FcγRIIb, the immunotherapeutic protein may comprise a binding domain such as an antigen binding region targeted to an activating Fc receptor or one or more subunits thereof such as an immunoglobulin Fc binding subunit or associated subunits required for expression and/or signalling (eg the binding domain can be targeted to any of the subunits of FcεRI, which is composed of the ligand binding chain FcεRIα subunit, as well as associated FcεRIβ and γ subunits) regardless of whether or not an immune complex is bound to the activating Fc receptor (or one or more subunits). As such, an immunotherapeutic protein targeted in this way can also be used for the treatment or prevention of allergic diseases (eg where an immune complex comprising the allergen is bound to the activating FcR), autoimmune diseases such as SLE and MS (eg where an immune complex comprising the autoantigen is bound to the activating FcR), other inflammatory diseases such as immune complex vasculitis (eg where an immune complex comprising the relevant antigen is bound to the activating FcR), antibody-mediated transplant rejection (eg where an immune complex comprising the antigen from a transplanted tissue or organ is bound to the activating FcR), and infectious diseases (eg where an immune complex comprising the antigen of an infectious agent is bound to the activating FcR).

Still further, in some examples where the immunotherapeutic protein is intended to be used for the treatment or prevention of a disease or condition by binding to and activating FcγRIIb, the immunotherapeutic protein may comprise a binding domain such as an antigen binding region targeted to an antigen of interest that is bound to the B cell receptor complex (BCR) (ie where the antigen is bound to membrane IgE, IgG or IgA on the surface of the B cell). As such, an immunotherapeutic protein targeted in this way can be used for the treatment or prevention of allergic diseases (eg where the immunotherapeutic protein binds to the antigen that is bound to the membrane immunoglobulin of a BCR) such that co-cross-linking of FcγRIIb to the BCR comprising the bound antigen results in the activation of FcγRIIb, thereby recruiting FcγRIIb inhibitory action to shut down antibody production (and/or B cell proliferation). Analogously, such an immunotherapeutic protein can be used for the treatment or prevention of autoimmune diseases such as SLE and MS, other inflammatory diseases such as immune complex vasculitis, antibody-mediated transplant rejection and infectious diseases.

Yet still further, in some examples where the immunotherapeutic protein is intended to be used for the treatment or prevention of a disease or condition by binding to and activating FcγRIIb, the immunotherapeutic protein may comprise a binding domain such as an antigen binding region targeted to an activating receptor that is other than an Fc receptor such as a B cell antigen receptor (BCR) complex. For example, the immunotherapeutic protein may be targeted to a membrane immunoglobulin of a BCR complex (eg membrane IgE, IgG or IgA on the surface of the B cell) by targeting, for example, the variable domain of the membrane immunoglobulin (eg the immunotherapeutic protein may be an anti-idiotypic IgG2 antibody). The membrane immunoglobulin of the targeted BCR may or may not comprise a bound antigen. As such, an immunotherapeutic protein targeted in this way can be used for the treatment or prevention of allergic diseases (eg where the immunotherapeutic protein binds to the membrane immunoglobulin of a BCR complex with or without bound allergen) such that co-cross-linking of FcγRIIb to the BCR results in the activation of FcγRIIb, thereby recruiting FcγRIIb inhibitory action to shut down antibody production (and/or B cell proliferation). Analogously, such an immunotherapeutic protein can be used for the treatment or prevention of autoimmune diseases such as SLE and MS, other inflammatory diseases such as immune complex vasculitis, antibody-mediated transplant rejection and infectious diseases. In addition, an immunotherapeutic protein targeted to a non-Fc type activating receptor such as a BCR, can also be used for the treatment or prevention of proliferative diseases, especially lymphoproliferative disorders (LPDs) such as leukaemias (eg acute lymphoblastic leukaemia (ALL) and chronic lymphocytic leukaemia (CLL)), lymphomas (eg B cell lymphomas and T cell lymphomas) and X-linked proliferative disease, wherein the ability of the immunotherapeutic protein to bring about co-cross-linking of FcγRIIb to the BCR can then cause activation of the FcγRIIb and thereby recruitment of FcγRIIb inhibitory action to shut down proliferation of cancerous cells.

Yet still further, in some examples where the immunotherapeutic protein is intended to be used for the treatment or prevention of a disease or condition by binding to and activating FcγRIIb, the immunotherapeutic protein may comprise a binding domain such as an antigen binding region targeted to a B cell antigen receptor (BCR) complex or one or more subunits thereof such as the membrane immunoglobulin or associated subunits required for expression and/or signalling (eg the binding domain can be targeted to any of the subunits of the BCR complex, which is composed of the antigen binding membrane immunoglobulin (eg IgM, IgD, IgG, IgE or IgA) as well as associated Ig-α or β chains (eg CD79a or CD79b) or other associated proteins (eg CD19, CD21 or CD81). The membrane immunoglobulin of the targeted BCR may or may not comprise a bound antigen. As such, an immunotherapeutic protein targeted in this way can also be used for the treatment or prevention of allergic diseases (eg where an allergen may or may not be bound to the BCR complex), autoimmune diseases such as SLE and MS (eg where an the autoantigen is bound to the BCR complex), other inflammatory diseases such as immune complex vasculitis (eg where the relevant antigen is bound to the BCR complex), antibody-mediated transplant rejection (eg where the relevant antigen from a transplanted tissue or organ is bound to the BCR complex), and infectious diseases (eg where the relevant antigen of an infectious agent is bound to the BCR complex).

Allergic Diseases

Mast cells and basophils, two key effector cells in the pathogenesis of allergic disorders, both express the high-affinity IgE receptor, FcεRI. However, they differ in their expression profile of the IgG receptors, FcγRs; basophils expressing the inhibitory receptor FcγRIIb, which is able to inhibit FcεRI signalling and decrease basophil activation. Since an immunotherapeutic protein of the present disclosure such as a mutant IgG2 antibody may be targeted to bind to an allergen (within an immune complex bound to an activating FcR on the surface of a basophil) and also bind to FcγRIIb to bring about co-cross-linking to the activating FcR to thereby activate the FcγRIIb (ie to recruit FcγRIIb inhibitory function; more specifically, binding of the mutant IgG2 antibody to FcγRIIb mediates FcγRIIb-dependent inhibition of allergic basophil activation by IgE), the method of the present disclosure enables the treatment or prevention of allergic diseases and conditions such as severe hay fever, atopic dermatitis, food allergies such as peanut allergy, and allergies to toxins (eg bee venom). Subsets of human mast cells to express FcγRIIb may also acts to regulate allergic diseases and conditions (eg food allergy, Burton O T et al., J Allergy Clin Immunol 141(1):189-201.e3, 2018) via its expression on subsets of human mast cell cells (Burton O T et al., Front Immunol 9:1244. doi: 10.3389/fimmu 2018). Thus, an immunotherapeutic protein of the present disclosure such as a mutant IgG2 antibody may recruit FcγRIIb inhibitory function on mast cells. Desirably, the mutant IgG2 antibody may also show no or poor binding ability to FcγRI (which can induce potent mast cell activation) to avoid unwanted mast cell activation.

Autoimmune Diseases and Conditions

By promoting activation of the FcγRIIb receptors that are present in affected subjects by, for example, co-cross-linking FcγRIIb to an activating type FcR through a bound immune complex comprising an autoantigen, the method of the present disclosure enables the beneficial treatment and/or prevention of autoimmune diseases and conditions such as those mentioned above. However, decreased expression and/or signalling activity of FcγRIIb in a subject (eg resulting from polymorphisms in the promoter and transmembrane domains of FcγRIIb that influence receptor expression and signalling) has been associated with increased susceptibility to autoimmune diseases and conditions, including systemic lupus erythematosus (SLE), Goodpasture syndrome, immune thrombocytopenia (ITP) and rheumatoid arthritis (RA) (Floto R A et al., Nat Med 11:1056-1058, 2005; Li X et al., Arthritis Rheum 48:3242-3252, 2003; and Radstake T R et al., Arthritis Rheum 54:3828-3837, 2006). Accordingly, in some embodiments where the method of the present disclosure is intended for use with a subject that is suffering from, or is predisposed to, an autoimmune disease or condition, the method may further comprise a step of selecting the subject by genotyping for relevant polymorphisms in the promoter and transmembrane domains of FcγRIIb (eg polymorphisms in the promotor region of FCGR2B (Su K et al., J Immunol 172:7186-7191, 2004; and Blank M C et al., Hum Genet 117:220-227, 2005), and an I232T polymorphism in the transmembrane domain of FcγRIIb (Kyogoku C et al., Arthritis Rheum 46:1242-1254, 2002)).

Infectious Diseases

An immunotherapeutic protein such as a mutant IgG2 antibody according to the present disclosure can be used to target the modulation of ITAM receptor-based signalling (ie by targeting ITAM receptors on cells co-expressing FcγRIIb (an ITIM receptor)) of, for example, the BCR on B lymphocytes. That is, in B cells, FcγRIIb inhibition of the BCR is a critical immune checkpoint for regulating antibody production (Lehmann B et al., Expert Rev Clin Immunol 8:243-254, 2012); possibly through the elimination by apoptosis of self-reactive B cells during somatic hyper-mutation (Pearse R N et al., Immunity 10:753-760, 1999) thereby constraining the selective antigen specificity of the humoral immune system and directing B cell production towards an appropriate antibody repertoire. This can, of course, be beneficial in “fighting” an infection by, for example, a bacterial pathogen (eg Neisseria meningitides, Streptococcus pneumoniae, Haemophilus influenzae and methicillin-resistant Staphylococcus aureus (“Golden Staph”)) or a virus (eg hepatitis C (HCV), and human immunodeficiency virus-1 (HIV-1)).

Proliferative Diseases

The BCR of B lymphocytes has been shown to be involved in the pathogenesis of various B cell-derived lymphoid cancers, and increasing amounts of evidence implicates antigen-independent self-association of BCRs as a key feature in a growing number of B cell neoplasia types such as chronic lymphocytic leukaemia (CLL), Heavy-chain diseases (HCDs) and activated B cell-like subtype diffuse large B cell lymphoma (ABC DLBCL) (Dühren-von Minden M et al., Nature 463(7415):309-312, 2012; Corcos D et al., Current Biology 5(10):1140-1148, 1995; and Davis R E et al., Nature 463(7277):88-92, 2010).

Accordingly, the method of the present disclosure may also be applied to the prevention and/or treatment of proliferative diseases and, particularly, B cell-derived lymphoid cancers, wherein the mutant IgG2 antibody can be used to activate FcγRIIb on B cells by, for example, co-cross-linking FcγRIIb to a BCR through a relevant antigen complexed to the BCR or a subunit of the BCR (ie targeted by the mutant IgG2 antibody), so as to bring about inhibition of BCR signalling.

Treatment of diseases through FcγRIIb-mediated endocytosis/internalisation (“sweeping”) In some other embodiments, the method of the present disclosure may be used for treating a disease or condition in a subject, wherein the clearance of immune complexes (e.g. “small” soluble complexes comprising, for example, opsonised virus, proteins (eg cytokines) and toxins; Iwayanagi Y et al., J Immunol 195: 3198-3205, 2015; and Mates J M et al., Front Immunol 8:35, 2017) from circulation by FcγRIIb present on the surface of leukocytes and some other non-haematopoietic cell types (eg liver sinusoidal endothelial cells (LSEC)) is beneficial in the treatment or prevention of said disease or condition. Mechanistically different from phagocytosis, which is performed by activating Fc receptor types and phagocytes (eg FcγRIII) and removes large immune complexes comprising, for example, large things such as bacteria, parasites and cancerous cells, there is some considerable interest in developing new therapeutics around this phenomenon (see, for example, Iwayanagi Y et al., 2015 supra; and Chenoweth A M et al., 2020 supra). Thus, in some examples of such embodiments, an immunotherapeutic protein such as a mutant IgG2 antibody may be targeted to, for example, an antigen (eg a viral antigen) or other protein or chemical entity such as an immunoglobulin, hormone, metabolite, cytokine or toxin to enable formation of a small soluble immune complex. Binding of the immunotherapeutic protein to an FcγRIIb receptor of LSEC may then mediate the sweeping clearance of the immune complex from the circulation. Therefore, such an immunotherapeutic protein can be used for the treatment or prevention of, for example, infectious diseases (eg. a viral infection such as an infection with the SARS-CoV-2 virus or an infection characterised by the production of toxins such as an endotoxin) and endocrine disorders such as Cushing's syndrome (Buliman et al., J. Med. Life 9:12-18, 2016) and inflammatory diseases characterised by, for example, overexpression of a cytokine such as TNF receptor-associated periodic syndrome (TRAPS) characterised by overexpression of IL-1β from circulating monocytes during disease flares (Bachetti T et al., Ann Rheum Dis 72:1044-1052, 2013), psoriasis characterised by, for example, the production of IL-17 or IL-23, and rheumatoid arthritis characterised by the production of cytokines such as TNF or IL-1β, autoimmune disease characterised by the production of autoantibodies, and allergic disease characterised by the production of IgE.

Treatment of Diseases Through FcγRIIb-Mediated Scaffolding

In some other embodiments, the method of the present disclosure may be used for treating a disease or condition in a subject, wherein enhanced agonistic function of an immunotherapeutic protein may be achieved through FcγRIIb “scaffolding”, wherein no signal is generated in the effector cell but “super cross-linking” of an opsonizing antibody (eg such as a mutant IgG2 antibody of the present disclosure) by the FcγRIIb on one cell generates a signal in a conjugated target cell that may lead to beneficial therapeutic effects such as, for example, induction of apoptosis or activation in agonistic expansion of cells and/or their secretion of cytokines (Chenoweth A M et al., 2020 supra). Thus, in some examples of such embodiments, an immunotherapeutic protein such as a mutant IgG2 antibody may be targeted to, for example, a cancer antigen present on the surface of a cancerous cell (eg CD20), or a cell surface antigen (eg CTLA4) present on an immune cell (eg a T cell). Therefore, such an immunotherapeutic protein can be used for the treatment or prevention of, for example, proliferative diseases and autoimmune diseases.

It will be apparent from the above, that an immunotherapeutic protein of the present disclosure, preferably a mutant IgG2 antibody, will typically comprise an antigen binding region which specifically binds to a target such as an antigen of interest or an immunoglobulin such as an antibody bound to an activating Fc receptor, an immunoglobulin of a BCR complex, or a subunit of an activating Fc receptor complex or BCR complex, etc. Thus, for example, where the method of the present disclosure is intended for use with a subject that is suffering from an allergic disease, the antigen binding region of a mutant IgG2 antibody according to the present disclosure may specifically bind to an antigen which is an allergen (eg the bee venom allergen, Api m 1, and the peanut allergens, Ara h 1, Ara h 2, Ara h 3 and Ara h 6).

Similarly, in the context of a method of the present disclosure intended for use with a subject that is suffering from, or is predisposed to, an autoimmune disease or condition, the antigen binding region of the mutant IgG2 antibody may specifically bind to an antigen which is an autoantigen (eg one of the common anti-Sm/RNP, anti-Ro/La and anti-dsDNA autoantigens of SLE). Further, where the method is intended for use in treating an infectious disease, the antigen binding region of the mutant IgG2 antibody may specifically bind to a pathogenic antigen (eg bound in a BCR complex) or otherwise, the membrane immunoglobulin of a BCR complex including the pathogenic antigen. Similarly, the antigen binding region of a mutant IgG2 antibody intended for use in treating a proliferative disease may specifically bind to a cancer antigen bound in a BCR complex or, alternatively, in a method involving FcγRIIb-mediated scaffolding, the antigen binding region of the mutant IgG2 antibody may specifically bind to a cancer antigen present on the surface of a cancerous cell (eg a cell surface antigen differentially expressed and/or present in cancer cells such as the CD20 and CD52 antigens found on the surface of CLL cells, and mucins (eg MUC-1) which are overexpressed in some breast and pancreatic cancers, to bring about apoptosis of the cancerous cell.

In some embodiments, a mutant IgG2 antibody suitable for use in the method of the present disclosure may be provided as an immunoconjugate wherein the antibody is conjugated to another molecule such as a molecule providing, for example, an additional ability to bind to an antigen of interest (eg a bispecific IgG2 antibody comprising an antigen binding region with a first binding specificity and which is linked to another molecule having a second binding specificity) or other functionality (eg a detectable molecule such as a dye or a molecule of therapeutic significance such as a complementary drug molecule).

In some other embodiments, a mutant IgG2 antibody suitable for use in the method of the present disclosure may be provided in a dimeric or multimeric form (eg with 3, 4, 5, 6 (hexameric) etc. copies of the mutant IgG2 antibody). Standard methodologies for producing dimeric and multimeric forms of antibodies (eg Stradomers™ or through self-association of Fc regions of adjacent antibody molecules) are well known to those skilled in the art (see, for example, Diebolder C A et al., Science 343(6176):1260-1263, 2014).

The method of the present disclosure will be typically applied to the treatment of a disease or condition in a human subject. However, the subject may also be selected from, for example, livestock animals (eg cows, horses, pigs, sheep and goats), companion animals (eg dogs and cats) and exotic animals (eg non-human primates, tigers, elephants etc).

In a second aspect, the present disclosure provides the use of an immunotherapeutic protein as defined in the first aspect, for treating a disease or condition wherein binding to and/or activation of FcγRIIb is beneficial, including, for example, allergic diseases, autoimmune diseases and conditions, infectious diseases and proliferative diseases.

In a third aspect, the present disclosure provides the use of an immunotherapeutic protein as defined in the first aspect, in the manufacture of a medicament for treating a disease or condition wherein activation of FcγRIIb is beneficial, including, for example, allergic diseases, autoimmune diseases and conditions, other inflammatory diseases, infectious diseases and proliferative diseases.

In a fourth aspect, the present disclosure provides a pharmaceutical composition or medicament comprising an immunotherapeutic protein as defined in the first aspect, and a pharmaceutically acceptable carrier, diluent and/or excipient.

In this specification, a number of terms are used which are well known to those skilled in the art. Nevertheless, for the purposes of clarity, a number of these terms are hereinafter defined.

As used herein, the term “IgG2 antibody” refers to any antibody comprising heavy chain polypeptides comprising an IgG2 constant heavy (CH) region (ie where the CH1, CH2 and CH3 domains are IgG2 CH1, CH2 and CH3 domains, each of which, independently, comprise an amino acid sequence that is either identical to the wild type sequence or shows at least 95%, preferably at least 98%, identity to a wild type sequence; as, for example, the wild type human sequences previously described in detail in Wines B D et al., 2016 supra) such that the antibody comprises an “IgG2 backbone” including an IgG2 Fc domain, and includes antibodies that comprise chimeric heavy chain polypeptides wherein, for example, the variable heavy chain (VH) region is derived from an antibody of another immunoglobulin class or subclass (eg IgA, IgE, IgG1 etc), which may or may not be derived from the same species as the IgG2 CH region (eg the IgG2 antibody may comprise heavy chain polypeptides comprising a human IgG2 CH region and a murine IgE VH region). Further, it is to be understood that an “IgG2 antibody” may comprise light chain polypeptides comprising a constant light (CL) region and/or variable light (VL) region derived from IgG2 or any other immunoglobulin class or subclass or wherein the light chain polypeptides are chimeric wherein one of said regions is derived from an antibody from one immunoglobulin class or subclass and the other of said regions is derived from an antibody from another immunoglobulin class or subclass (eg the IgG2 antibody may comprise light chain polypeptides comprising a human Igκ CL region and an IgE-derived VL region).

The term “target” and derivatives thereof such as “targeted” and “targeting” will be well understood by those skilled in the art by the context in which the terms are used. For instance, a “target” will be understood as referring to something at which an action or process is directed; for example, a “target antigen” as used herein in the context of a characteristic/activity of an antibody will be understood as referring to an antigen to which that antibody binds, and similarly, by “targeting” something, it will be understood that an antibody (or other immunotherapeutic molecule) is prepared so as to bind to that something (eg an antigen, immune checkpoint, receptor etc).

As used herein, the term “% identity” between two amino acid sequences refers to sequence identity percentages understood as having been calculated using a mathematical algorithm such as that described by Karlin S and S F Altschul, Proc Natl Acad Sci USA 87:2264-2268, 1990, and as modified as in Karlin S and S F Altschul, Proc Natl Acad Sci USA 90:5873-5877, 1993. Such an algorithm is incorporated into the NBLAST and XBLAST programs of Altschul S F et al., J Mol Biol 215:403-410, 1990. BLAST protein searches can be performed with the XBLAST program using the default parameters (see ncbi.nlm.nih.gov/BLAST/). To determine the sequence identity percentage between two amino acid sequences, the mathematical algorithm may align the sequences for optimal comparison purposes, and calculate the percent identity between the sequences as a function of the number of identical positions shared by the sequences (ie percent identity=number of identical positions/total number of positions (eg overlapping positions)×100).

As used herein, the term “treating” includes prophylaxis as well as the alleviation of established symptoms of a disease or condition. As such, the act of “treating” a disease or condition therefore includes: (1) preventing or delaying the appearance of clinical symptoms of the disease or condition developing in a subject suffering from, or predisposed to, the disease or condition; (2) inhibiting the disease or condition (ie arresting, reducing or delaying the development of the disease or condition or a relapse thereof, in case of a maintenance treatment, or at least one clinical or subclinical symptom thereof); and (3) relieving or attenuating the disease or condition (ie causing regression of the disease or condition or at least one of clinical or subclinical symptom thereof).

As used herein, the phrase “manufacture of a medicament” includes the use of one or more immunotherapeutic protein as defined in the first aspect directly as the medicament or in any stage of the manufacture of a medicament comprising one or more immunotherapeutic protein as defined in the first aspect.

The term “effective amount” is an amount sufficient to effect beneficial or desired clinical results. An effective amount can be administered in one or more administrations. Typically, an effective amount is sufficient for treating a disease or condition or otherwise to palliate, ameliorate, stabilise, reverse, slow or delay the progression of a disease or condition. By way of example only, an effective amount of an immunotherapeutic protein such as a mutant IgG2 antibody may comprise between about 0.1 and about 250 mg/kg body weight per day, more preferably between about 0.1 and about 100 mg/kg body weight per day and, still more preferably between about 0.1 and about 25 mg/kg body weight per day. However, notwithstanding the above, it will be understood by those skilled in the art that an effective amount may vary and depend upon a variety of factors including the age, body weight, sex and/or health of the subject being treated, the activity of the particular protein, the metabolic stability and length of action of the particular protein, the route and time of administration of the particular protein, the rate of excretion of the particular protein and the severity of, for example, the disease or condition being treated.

The immunotherapeutic protein may be administered in combination with one or more additional agent(s) for the treatment of the particular disease or condition being treated. For example, the immunotherapeutic protein may be used in combination with other agents for treating allergic diseases (eg an antihistamine drug (including those administered intravenously (iv), cortisone and/or a beta-agonist drug such as albuterol)), or in the context of treating proliferative diseases, the immunotherapeutic protein may be used in combination with other agents for treating cancer (including, for example, antineoplastic drugs such as cis-platin, gemcitabine, cytosine arabinoside, doxorubicin, epirubicin, taxoids including taxol, topoisomerase inhibitors such as etoposide, cytostatic agents such as tamoxifen, aromatase inhibitors (eg as anastrozole) and inhibitors of growth factor function (eg antibodies such as the anti-erbB2 antibody trastuzumab (Herceptin™)).

Where used in combination with other agents, the immunotherapeutic protein can be administered in the same pharmaceutical composition or in separate pharmaceutical compositions. If administered in separate pharmaceutical compositions, the immunotherapeutic protein and the other agent(s) may be administered simultaneously or sequentially in any order (eg within seconds or minutes or even hours (eg 2 to 48 hours)).

The immunotherapeutic protein may be formulated into a pharmaceutical composition with a pharmaceutically acceptable carrier, diluent and/or excipient. Examples of suitable carriers and diluents are well known to those skilled in the art, and are described in, for example, Remington's Pharmaceutical Sciences, Mack Publishing Co., Easton, P A 1995. Examples of suitable excipients for the various different forms of pharmaceutical compositions described herein may be found in the Handbook of Pharmaceutical Excipients, 2nd Edition, (1994), Edited by A Wade and P J Weller. Examples of suitable carriers include lactose, starch, glucose, methyl cellulose, magnesium stearate, mannitol, sorbitol and the like. Examples of suitable diluents include ethanol, glycerol and water. The choice of carrier, diluent and/or excipient may be made with regard to the intended route of administration and standard pharmaceutical practice.

A pharmaceutical composition comprising an immunotherapeutic protein as defined in the first aspect may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, and even dyes may be provided in the pharmaceutical composition. Examples of preservatives include sodium benzoate, sorbic acid and esters of p-hydroxybenzoic acid. Anti-oxidants and suspending agents may be also used.

A pharmaceutical composition comprising an immunotherapeutic protein as defined in the first aspect will typically be adapted for intravenous or subcutaneous administration. As such, a pharmaceutical composition may comprise solutions or emulsions which may be injected into the subject, and which are prepared from sterile or sterilisable solutions. A pharmaceutical composition may be formulated in unit dosage form (ie in the form of discrete portions containing a unit dose, or a multiple or sub-unit of a unit dose).

The method, uses and pharmaceutical composition of the present disclosure are hereinafter further described with reference to the following, non-limiting example.

### EXAMPLES

### Example 1

### Methods and Materials

Antibodies and Reagents

F(ab′)2 fragments of rabbit anti-human IgE (Dako Agilent, Santa Clara, CA<United States of America) were produced by pepsin digest as described in Current Protocols in Immunology, Andrew S M and J A Titus, Chapter 2:Unit 2.8, 2001. Briefly, the rabbit antibody was dialysed against digest buffer (0.2 M NaOAc, pH 4.0) then an equal volume of pepsin (0.1 mg/ml) (Sigma-Aldrich, St Louis, MO, United States of America) in digest buffer added and incubated overnight at 37° C. Digestion was stopped by the addition of 2 M Tris base pH 8.0 10% (v/v) and then the digest dialysed against phosphate buffered saline (PBS, pH 8.0). The hapten 2,4,6-trinitrophenyl (TNP) was conjugated to F(ab′)2 fragments of rabbit anti-human IgE (anti-IgE-TNP), bee venom allergen (Api m 1-TNP) or to bovine serum albumin (BSA-TNP), by incubation with 10% 2,4,6-trinitrobenzene sulfonic acid in water (Sigma-Aldrich), diluted 1/20 in 0.1 M borate, pH 7.0, at room temperature for 90 minutes and then dialysed against PBS, pH 7.0.

Human Donors

Healthy donors and allergic patients were recruited and blood samples collected. Patients that had presented with honey bee venom allergy were tested for IgE reactivity to the relevant allergen by ImmunoCAP (Phadia, Uppsala, Sweden).

Expression of Cell Surface FcγR

Human FcγR were expressed in in the mouse B cell line IIA1.6, which lacks endogenous mouse Fc receptors. The cells expressing human FcγRIIa-H131, FcγRIIa-R131 and FcγRIIb, have been described previously (eg Powell M S et al., J Immunol 176(12):7489-7494, 2006; Ramsland P A et al., J Immunol 187(6):3208-3217, 2011; and Trist H M et al., J Immunol 192(2):792-803, 2014). Cell lines expressing human FcγRIIIa (GenBank:accession NP_001121065) allelic forms V158 and F158 and human FcγRI (Allen J M and B Seed, Science 243(4889):378-381, 1989) were generated as described for FcγRIIa (Powell et al., 2006 supra). Briefly, receptor cDNAs were separately cloned into the Gateway entry plasmid pENTR1A (Invitrogen Corporation, Waltham, MA, United States of America) using standard molecular biology techniques, followed by Gateway L R cloning into a Gateway-adapted pMXI expression vector containing a neomycin resistance cassette (Wines B et al., J Biol Chem 279(25):26339-26345, 2004). Retroviruses were generated using the Phoenix packaging cell line (Powell et al., 2006 supra) and used to transduce the IIA1.6 cell line for the expression of FcγRIIIa or FcγRI. IIA1.6 cells already expressing the human FcRγ chain (Wines et al., 2004 supra) were then transduced with either the FcγRI or FcγRIII retrovirus in order to produce the cells lines co-expressing FcRγ/FcγRI or FcRγ/FcγRIII. Expression of the receptors on the transduced cell populations was evaluated using biotinylated Fab or F(ab′)2 fragments of receptor specific antibodies, and streptavidin-APC (1/500). The anti-Fc receptor antibodies used were: FcγRIIa (IV-3 Fab-Biotin fragments), FcγRIIb (H2B6 F(ab′)2-Biotin), FcγRI (32.2 F(ab′)2-biotin), FcγRIIIa (3G8 F(ab′)2-biotin) and FcRγ-EGFP (green fluorescence protein), and were detected in the FITC channel.

Production of anti-TNP human IgG and mutant anti-TNP human IgG plasmid constructs Chimeric anti-TNP human IgG antibody constructs consisting of the variable heavy (VH) and light (VL) region sequence of the mouse anti-trinitrophenyl (anti-TNP) antibody TIB142 and the sequence from the constant heavy (CH) region from human IgG subclasses have been described previously in detail—IgG1 (Patel D et al., J Immunol 184(11):6283-6292, 2010), hIgG2 and hIgG4 (Wines B D et al., 2016 supra). This includes the stabilisation of the core hinge of IgG4-based mutants by conversion of the CPSC sequence of normal IgG4 to CPPC which prevents half molecule exchange. All chimeric antibody sequences were subcloned into the pCR3 vector. Mutations were made into the IgG constant heavy chain (CH) cDNA sequence using standard molecular biology techniques and are listed in Table 1.

TABLE 1
Summary of IgG mutants

| | | Mutation site* | | | | |
|---|---|---|---|---|---|---|
| Backbone | Nomenclature | 226-229 | 232-237 | 267 | 328 | Notes |
| IgG1 | IgG1-WT | CPPC | PELLGG | S | L | |
| | IgG1-SELF | CPPC | PELLGG | E | F | |
| IgG2 | IgG2-WT | CPPC | PPVAG | S | L | |
| | IgG2-LLGG | CPPC | PELLGG | S | L | IgG1 like lower hinge |
| | IgG2-FEGG | CPPC | PEFEGG | S | L | |
| | IgG2-FEGG-SELF | CPPC | PEFEGG | E | F | |
| | IgG2-FLGG | CPPC | PEFLGG | S | L | IgG4 like lower hinge |
| | IgG2-FLGG-SELF | CPPC | PEFLGG | E | F | IgG4 like lower hinge |
| IgG4 | IgG4-WT | CPSC | PEFLGG | S | L | |
| | IgG4-LLGG | CPPC | PELLGG | S | L | IgG1 like lower hinge |
| | IgG4-SELF | CPSC | PEFLGG | E | F | |
| | IgG4-FEGG | CPPC | PEFEGG | S | L | |
| | IgG4-FEGG-SELF | CPPC | PEFEGG | E | F | |

*Sequence numbering is based on human IgG1 Eu number. Mutations are in bold and underlined. Lower hinge sequences (232-237) are italicised. Sequence listing numbers are as follows: CPPC (SEQ ID NO: SEQ ID NO: 9), PELLGG (SEQ ID NO: 10), PPVAG (SEQ ID NO: 11), PEFEGG (SEQ ID NO: 12), and PEFLGG (SEQ ID NO: 13).

cDNA sequences encoding the constant region of the IgG heavy chain polypeptides (CH) for IgG2-FEGG-SELF and IgG2-FLGG-SELF are provided in Table 2, shown as ligated to the cDNA sequence for the variable heavy (VH) region. The cDNA sequence for the variable light (VL) region of the mouse (anti-TNP) antibody is also provided.

TABLE 2

| | cDNA sequence and amino acid translation | |
|---|---|---|
| Anti-TNP mAb formatted as | 1 | AAG CTT ACC ATG GTG CTT AGT TTG CTG TAT |
| IgG2-FEGG-SELF mutant | | M V L S L L Y |
| Bold and underlined amino | 31 | CTG CTC ACC GCC CTG CCT GGA ATC CTG AGC L L |
| acid sequences correspond to | | T A L P G I L S |
| the core hinge sequence (aa | 61 | GAG GTG CAG TTG CAG GAA TCT GGT CCC AGT E V |
| residues 226-229, according | | Q L Q E S G P S |
| to Eu numbering convention) | 91 | CTA GTT AAG CCC AGC CAG ACA CTG AGC CTG L V |
| and the portion of the lower | | K P S Q T L S L |
| hinge sequence (aa | 121 | ACA TGT AGT GTG ACC GGT GAC AGC ATT ACA T C |
| residues | | S V T G D S I T |
| 232-237) and mutations | 151 | AGC GGC TAC TGG AAC TGG ATC AGA CAG GTG S G |
| S267E and L328F as | | Y W N W I R Q V |
| disclosed in Table 1. The c- | 181 | CCA GGA AAC AAA CTC GAA TAC ATG GGG TTC P G |
| terminal sequence LGK | | N K L E Y M G F |
| results from subcloning. | 211 | ATC AAT TAC AGT GGC AAT ACT TAC TAT AAT I N |
| | | Y S G N T Y Y N |
| | 241 | CCC AGC CTG AGA AGC AGA ATT TCT ATA ACC P S |
| | | L R S R I S I T |
| | 271 | AGA GAC ACC AGC AAA AAC CAG TAC TTT CTG R D |
| | | T S K N Q Y F L |
| | 301 | CAC CTG AAC AGC GTA ACA ACG GAA GAC ACC H L |
| | | N S V T T E D T |
| | 331 | GCC ACC TAT TAC TGC GCC AGG GCC AAC TGG A T |
| | | Y Y C A R A N W |
| | 361 | GAT GTG TTC GCA TAC TGG GGC CAG GGC ACT D V |
| | | F A Y W G Q G T |
| | 391 | CTG GTG ACG GTG AGC GCC GCG TCG ACA AAA L V |
| | | T V S A A S T K |
| | 421 | GGC CCT AGC GTC TTT CCA CTC GCC CCA TGT G P |
| | | S V F P L A P C |
| | 451 | TCA AGA AGC ACC AGC GAG TCA ACT GCG GCT S R |
| | | S T S E S T A A |
| | 481 | TTG GGC TGC TTG GTG AAG GAT TAC TTC CCA L G |
| | | C L V K D Y F P |
| | 511 | GAG CCT GTC ACT GTC AGC TGG AAT AGC GGG |
| | | E P V T V S W N S |
| | 541 | GC CT AC AG GG GT CA AC TTTCCC |
| | | T G C T A G C C |
| | | A L T S G V H T F P |
| | 571 | GC GT TTACA AG AG GG CTCTATAGC |
| | | C G G C C C |
| | | A V L Q S S G L Y S |
| | 601 | CT AG AG GT GT AC GT CC TC AGC |
| | | G C T G A T G C G |
| | | L S S V V T V P S S |
| | 631 | AA TTTGG AC CA AC TATAC TG AAT |
| | | T T C G C A C |
| | | N F G T Q T Y T C N |
| | 661 | GT GA CA AA CC AG AA AC AA GTA |
| | | C T T A C T C A G |
| | | V D H K P S N T K V |
| | 691 | GA AA AC GT GA AG AA TG TGTGTG |
| | | C G A C A A A C |
| | | D K T V E R K C C V |
| | 721 | GA TG CCTCCTTG CC GC CC GA TTT |
| | | G C C C C C G |
| | | E C P P C P A P E F |
| | 751 | GA GG GG CCTTC GTTTTTCTTTTTCCA |
| | | G G G G |
| | | E G G P S V F L F P |
| | 781 | CCTAA CC AA GA AC CT AT AT AGC |
| | | A G A C C G G C |
| | | P K P K D T L M I S |
| | 811 | AG AC CC GA GT AC TGTGT GT GTG |
| | | G A A A G A C C |
| | | R T P E V T C V V V |
| | 841 | GA GT GA CA GA GA CC GA GT CAA |
| | | C A G T A C G G G |
| | | D V E H E D P E V Q |
| | 871 | TTCAA TG TATGTTGA GG GT GA GTG |
| | | C G T T G A |
| | | F N W Y V D G V E V |
| | 901 | CA AA GC AA AC AA CC CG GA GAA |
| | | T T A A T G A G G |
| | | H N A K T K P R E E |
| | 931 | CA TTCAA AG AC TTCAG GT GTTAGC |
| | | G T C A A A |
| | | Q F N S T F R V V S |
| | 961 | GTTCT AC GT GT CA CA GA TG CTG |
| | | G T C G C A T G |
| | | V L T V V H Q D W L |
| | 991 | AA GG AA GA TA AA TGTAA GT AGC |
| | | C G G A C G G G |
| | | N G K E Y K C K V S |
| | 1021 | AA AA GG TTTCC GC CC ATTGA AAA |
| | | C A T C T A G |
| | | N K G F P A P I E K |
| | 1051 | AC ATTTCTAA AC AA GG CA CC CGG |
| | | A G C A A G C |
| | | T I S K T K G Q P R |
| | 1081 | GA CC CA GT TATAC CT CC CCTAGC |
| | | G A G G T G G |
| | | E P Q V Y T L P P S |
| | 1111 | AG GA GA AT AC AA AA CA GT AGC |
| | | G G A G C G C G G |
| | | R E E M T K N Q V S |
| | 1141 | CT AC TGTCT GT AA GG TTCTA CCC |
| | | G C G G A C C |
| | | L T C L V K G F Y P |
| | 1171 | AG GA AT GC GTTGA TG GA AG AAT |
| | | C T C A G G G C |
| | | S D I A V E W E S N |
| | 1201 | GG CA CC GA AA AA TA AA AC ACG |
| | | C A C G C C C G T |
| | | G Q P E N N Y K T |
| | 1231 | CCC CCC ATG CTT GAT AGC GAC GGA TCA TTT |
| | | P P M L D S D G S F |
| | 1261 | TTC CTG TAC TCA AAA CTG ACC GTG GAC AAA |
| | | F L Y S K L T V D K |
| | 1291 | AGC AGA TGG CAG CAG GGA AAC GTT TTC AGT |
| | | S R W Q Q G N V F S |
| | 1321 | TGC TCA GTG ATG CAC GAA GCG CTG CAC AAT |
| | | C S V M H E A L H N |
| | 1351 | CAT TAT ACT CAG AAA AGC CTG AGC TTG AGC |
| | | H Y T Q K S L S L S |
| | 1381 | TTA GGA AAA TGA TCT AGA (SEQ ID NO: 15) |
| | | L G K * (SEQ ID NO: 16) |
| Anti-TNP mAb | 1 | AAG CTT ACC ATG GTG CTT AGT TTG CTG TAT K L |
| formatted as IgG2- | | T M V L S L L Y |
| FLGG-SELF mutant | 31 | CTG CTC ACC GCC CTG CCT GGA ATC CTG AGC L L |
| Bold and underlined amino | | T A L P G I L S |
| acid sequences correspond to | 61 | GAG GTG CAG TTG CAG GAA TCT GGT CCC AGT E V |
| the corehinge sequence (aa | | Q L Q E S G P S |
| residues 226-229, according | 91 | CTA GTT AAG CCC AGC CAG ACA CTG AGC CTG L V |
| to Eu numbering convention) | | K P S Q T L S L |
| and the portion of the | 121 | ACA TGT AGT GTG ACC GGT GAC AGC ATT ACA T C |
| lower hinge sequence (aa | | S V T G D S I T |
| residues 232-237) and | 151 | AGC GGC TAC TGG AAC TGG ATC AGA CAG GTG S G |
| mutations | | Y W N W I R Q V |
| S267E and L328F as | 181 | CCA GGA AAC AAA CTC GAA TAC ATG GGG TTC P G |
| disclosed in | | N K L E Y M G F |
| Table 1. | 211 | ATC AAT TAC AGT GGC AAT ACT TAC TAT AAT I N |
| | | Y S G N T Y Y N |
| | 241 | CCC AGC CTG AGA AGC AGA ATT TCT ATA ACC P S |
| | | L R S R I S I T |
| | 271 | AGA GAC ACC AGC AAA AAC CAG TAC TTT CTG R D |
| | | T S K N Q Y F L |
| | 301 | CAC CTG AAC AGC GTA ACA ACG GAA GAC ACC H L |
| | | N S V T T E D T |
| | 331 | GCC ACC TAT TAC TGC GCC AGG GCC AAC TGG A T |
| | | Y Y C A R A N W |
| | 361 | GAT GTG TTC GCA TAC TGG GGC CAG GGC ACT D V |
| | | F A Y W G Q G T |
| | 391 | CTG GTG ACG GTG AGC GCC GCG TCG ACA AAA L V |
| | | T V S A A S T K |
| | 421 | GGC CCT AGC GTC TTT CCA CTC GCC CCA TGT G P |
| | | S V F P L A P C |
| | 451 | TCA AGA AGC ACC AGC GAG TCA ACT GCG GCT |
| | | S R S T S E S T A A |
| | 481 | TTGGG TG TTGGT AA GA |
| | | C C G G T C |
| | | L G C L V K D Y F P |
| | 511 | GA CCTGT AC GT AG TG AA AG GGG |
| | | G C T C C G T C |
| | | E P V T V S W N S G |
| | 541 | GC CT AC AG GG GT CA AC TTTCCC |
| | | T G C T A G C C |
| | | A L T S G V H T F P |
| | 571 | GC GT TTACA AG AG GG CTCTATAGC |
| | | C G G C C C |
| | | A V L Q S S G L Y S |
| | 601 | CT AG AG GT GT AC GT CC TC AGC |
| | | G C T G A T G C G |
| | | L S S V V T V P S S |
| | 631 | AA TTTGG AC CA AC TATAC TG AAT |
| | | T T C G C A C |
| | | N F G T Q T Y T C N |
| | 661 | GT GA CA AA CC AG AA AC AA GTA |
| | | C T T A C T C A G |
| | | V D H K P S N T K V |
| | 691 | GA AA AC GT GA AG AA TG TGTGTG |
| | | C G A C A A A C |
| | | D K T V E R K C C V |
| | 721 | GA TG CCTCCTTG CC GC CC GA TTT |
| | | G C C C C C G |
| | | E C P P C P A P E F |
| | 751 | CT GG GG CCTTC GTTTTTCTTTTTCCA |
| | | G G G G |
| | | L G G P S V F L F P |
| | 781 | CCTAA CC AA GA AC CT AT AT AGC |
| | | A G A C C G G C |
| | | P K P K D T L M I S |
| | 811 | AG AC CC GA GT AC TGTGT GT GTG |
| | | G A A A G A C C |
| | | R T P E V T C V V V |
| | 841 | GA GT GA CA GA GA CC GA GT CAA |
| | | C A G T A C G G G |
| | | D V E H E D P E V Q |
| | 871 | TTCAA TG TATGTTGA GG GT GA GTG |
| | | C G T T G A |
| | | F N W Y V D G V E V |
| | 901 | CA AA GC AA AC AA CC CG GA GAA |
| | | T T A A T G A G G |
| | | H N A K T K P R E E |
| | 931 | CA TTCAA AG AC TICAG GT GTTAGC |
| | | G T C A A A |
| | | Q F N S T F R V V S |
| | 961 | GTTCT AC GT GT CA CA GA TG CTG |
| | | G T C G C A T G |
| | | V L T V V H Q D W L |
| | 991 | AA GG AA GA TA AA TGTAA GT AGC |
| | | C G G A C G G G |
| | | N G K E Y K C K V S |
| | 1021 | AA AA GG TTTCC GC CC ATTGA AAA |
| | | C A T C T A G |
| | | N K G F P A P I E K |
| | 1051 | AC ATTTCTAA AC AA GG CA CC CGG |
| | | A G C A A G C |
| | | T I S K T K G Q P R |
| | 1081 | GA CC CA GT TATAC CT CC CCTAGC |
| | | G A G G T G G |
| | | E P Q V Y T L P P S |
| | 1111 | AG GA GA AT AC AA AA CA GT AGC |
| | | G G A G C G C G G |
| | | R E E M T K N Q V S |
| | 1141 | CT AC TGTCT GT AA GG TTCTA CCC |
| | | G C G G A C C |
| | | L T C L V K G F Y P |
| | 1171 | AGC GAT ATC GCA GTT GAG TGG GAG AGC AAT S D |
| | | I A V E W E S N |
| | 1201 | GGC CAA CCC GAG AAC AAC TAC AAG ACT ACG G |
| | | Q P E N N Y K T T |
| | 1231 | CCC CCC ATG CTT GAT AGC GAC GGA TCA TTT P P |
| | | M L D S D G S F |
| | 1261 | TTC CTG TAC TCA AAA CTG ACC GTG GAC AAA F L |
| | | Y S K L T V D K |
| | 1291 | AGC AGA TGG CAG CAG GGA AAC GTT TTC AGT S R |
| | | W Q Q G N V F S |
| | 1321 | TGC TCA GTG ATG CAC GAA GCG CTG CAC AAT C S |
| | | V M H E A L H N |
| | 1351 | CAT TAT ACT CAG AAA AGC CTG AGC TTG AGC H Y |
| | | T Q K S L S L S |
| | | 1381 TTA GGA AAA TGA TCT AGA (SEQ ID NO: 17) |
| | | L G K * (SEQ ID NO: 18) |
| Chimeric anti-TNP light | 1 | ATG GAT TCC CAG GCC CAG GTG CTC ATG CTG M D |
| chain | | S Q A Q V L M L |
| Codon optimised nucleotide | 31 | TTA CTC CTG TGG GTC TCC GGA ACC TGC GGC L L |
| sequence with mouse VH | | L W V S G T C G |
| joined to human constant | 61 | GAC ATA GTG ATG TCA CAG AGC CCA AGC AGC D I |
| kappa light chain domain. | | V M S Q S P S S |
| | 91 | CTT GCA GTG TCT GTT GGT GAG AAG GTG ACC L A |
| | | V S V G E K V T |
| | 121 | ATG AGC TGC AAG TCA AGC CAG AGC CTG CTC M S |
| | | C K S S Q S L L |
| | 151 | TAT AGC AGC AAT CAG AAG AAC TAT TTG GCC Y S |
| | | S N Q K N Y L A |
| | 181 | TGG TAT CAG AGA AAA CCC GGC CAG AGC CCT W Y |
| | | Q R K P G Q S P |
| | 211 | AAG CTC CTT ATT TAT TGG GCT AGC ACA CGC K L |
| | | L I Y W A S T R |
| | 241 | GAG TCC GGC GTG CCG GAC AGA TTT ACC GGC E S |
| | | G V P D R F T G |
| | 271 | AGC GGT AGC GGC ACC GAT TTT ACT TTG ACC S G |
| | | S G T D F T L T |
| | 301 | ATC TCG TCT GTA AAG GCC GAA GAC CTG GCA I S |
| | | S V K A E D L A |
| | 331 | GTG TAT TAC TGT CAA CAT TAT TAC TCA TCT |
| | | V Y Y C Q H Y Y S S |
| | 361 | CCC TAC ACT TTC GGA GGG GGG ACC AAA CTG P Y |
| | | T F G G G T K L |
| | 391 | GAA ATT AAG CGT ACG GTT GCT GCC CCT TCT E I |
| | | K R T V A A P S |
| | 421 | GTC TTC ATC TTC CCT CCC AGC GAT GAA CAG |
| | | V F I F S D E Q |
| | | P P |
| | 451 | CTG AAA AGT GGG ACA AG GT GT TGC |
| | | GCG T A G |
| | | L K S G S V V C |
| | | T A |
| | 481 | CTG CTA AAC AAT TTT CC CG GA GCC |
| | | TAC A G G |
| | | L L N N P R E A |
| | | F Y |
| | 511 | AAA GTG CAG TGG AAA GA AA GC CTG |
| | | GTG C C T |
| | | K V Q W D N A L |
| | | K V |
| | 541 | CAA AGT GGA AAT TCT GA TCCGT ACA |
| | | CAG G C |
| | | Q S G N E S V T |
| | | S Q |
| | 571 | GAG CAG GAC TCG AAG AG AC TA AGC |
| | | GAC C T C |
| | | E Q D S S T Y S |
| | | K D |
| | 601 | TTG TCA AGC ACC CTG CT AG AA GCC |
| | | ACG G C A |
| | | L S S T L S K A |
| | | L T |
| | 631 | GAT TAC GAG AAG CAC GT TA GC TGC |
| | | AAG G C A |
| | | D Y E K V Y A C |
| | | H K |
| | 661 | GAA GTT ACG CAC CAA CT AG AG CCA |
| | | GGA G T C |
| | | E V T H L S S P |
| | | Q G |
| | 691 | GTC ACA AAG AGC TTC AG GG GA TGT |
| | | AAC G G A |
| | | V T K S R G E C |
| | | F N |
| | 721 | TAG (SEQ ID NO: 19) |
| | | * (SEQ ID NO: 20) |

Production of Anti-Human IgE and Mutant Anti-Human IgE Plasmid Constructs

Anti-human IgE antibody constructs comprised synthetic DNA encoding the variable heavy (VH) region and light chain region sequences of the therapeutic mAb, omalizumab (ThermoFisher, GeneArt, Waltham, MA, United States of America). H chain constructs comprised the VH cDNA sequence and constant domain sequence for IgG4 and IgG2 variants. DNAs for IgG heavy and light chains were subcloned into the expression vector pCR3 or pcDNA3.4. Mutations in the IgG2 constant heavy chain (CH) cDNA sequence affecting binding are listed in Table 1.

cDNA sequences encoding the constant region of the IgG heavy chain polypeptides (CH) or anti-IgE light chain (CL), IgG4 and IgG2-FEGG-SELF and IgG2-FLGG-SELF are provided in Table 3, shown as ligated to the cDNA sequence for the variable heavy (VH) region. The cDNA sequence for the anti-IgE antibody light chain is also provided.

TABLE 3

| | cDNA sequence and amino acid translation | |
|---|---|---|
| Anti-IgE formatted as IgG4 | 1 | ATG GGC TGG TCC TGC ATC ATC CTG TTT CTG |
| Bold and underlined amino | | M G W S C I I L F L |
| acid sequences correspond to | 31 | GTG GCC ACA GCC ACC GGC GTG CAC TCT GAA |
| the core hinge sequence (aa | | V A T A T G V H S E |
| residues 226- 229, according | 61 | GTG CAG CTG GTG GAA TCT GGC GGC GGA CTG |
| to Eu numbering convention) | | V Q L V E S G G G L |
| and the portion of the lower | 91 | GTG CAG CCT GGC GGA TCT CTG AGA CTG AGC |
| hinge sequence (aa residues | | V Q P G G S L R L S |
| 232-237) and mutations | 121 | TGT GCC GTG TCC GGC TAC AGC ATC ACC AGC |
| S267E and L328F as | | C A V S G Y S I T S |
| disclosed in Table 1. | 151 | GGC TAC TCT TGG AAC TGG ATC CGG CAG GCC |
| | | G Y S W N W I R Q A |
| | 181 | CCT GGC AAG GGA CTG GAA TGG GTG GCC TCC |
| | | P G K G L E W V A S |
| | 211 | ATC ACC TAC GAC GGC AGC ACC AAC TAC AAC |
| | | I T Y D G S T N Y N |
| | 241 | CCC AGC GTG AAG GGC CGG ATC ACC ATC AGC |
| | | P S V K G R I T I S |
| | 271 | AGG GAC GAC AGC AAG AAC ACA TTC TAC CTG |
| | | R D D S K N T F Y L |
| | 301 | CAA ATG AAC AGC CTG CGG GCC GAG GAC ACC |
| | | Q M N S L R A E D T |
| | 331 | GCC GTG TAC TAT TGT GCC AGA GGC AGC CAC |
| | | A V Y Y C A R G S H |
| | 361 | TAC TTC GGC CAC TGG CAC TTT GCC GTG TGG |
| | | Y F G H W H F A V W |
| | 391 | GGC CAG GGA ACA ACG GTC ACC GTG TCT GCA |
| | | G Q G T T V T V S A |
| | 421 | GCG TCG ACA AAA GGT CCC AGC GTG TTT CCC |
| | | A S T K G P S V F P |
| | 451 | CTG GCT CCT TGT AGC AGA AGC ACG TCA GAA |
| | | L A P C S R S T S E |
| | 481 | AGT ACA GCT GCC CTG GGA TGC CTG GTG AAA |
| | | S T A A L G C L V K |
| | 511 | GAT TAT TTT CCC GAG CCC GTT ACC GTT TCC |
| | | D Y F P E P V T V S |
| | 541 | TGG AAT AGT GGC GCC CTG ACC TCG GGC GTG |
| | | W N S G A L T S G V |
| | 571 | CAT ACT TTT CCC GCT GTT CTG CAG AGC AGC |
| | | H T F P A V L Q S S |
| | 601 | GGC CTG TAT AGC CTG AGC AGT GTG GTG ACT |
| | | G L Y S L S S V V T |
| | 631 | GTT CCG TCT AGC AGC CTG GGT ACC AAG ACT |
| | | P S S S L G T K T |
| | 661 | TAT ACA TGC AAC GTG GAC CAT AAG CCT AGC |
| | | T C N V D H K P S |
| | 691 | AAT ACT AAG GTA GAT AAG CGA GTG GAG AGC |
| | | N T K V D K R V E S |
| | 721 | AAG TAC GGC CCC CCC TGC CCT AGC TGT CCC |
| | | K Y G P P C P S C P |
| | 751 | GC CCTGA TTTCT GG GG CC AGC GTG |
| | | A G A C G C |
| | | A P E F L G G P S V |
| | 781 | TTTCT TTCCC CC AA CCTAA GAT ACC |
| | | G C G G A |
| | | F L F P P K P K D T |
| | 811 | CT AT AT AG AG AC CC GA GTG ACC |
| | | G G C C A A C A |
| | | L M I S R T P E V T |
| | 841 | TG GT GTTGT GA GT AG CA GAA GAT |
| | | C G G C C C G |
| | | C V V V D V S Q E D |
| | 871 | CCTGA GT CA TTCAA TG TA |
| | | G G G T G C |
| | | P E V Q F N W Y V D |
| | 901 | GG GT GA GT CA AA GC AA ACA AAG |
| | | C G G G C T T G |
| | | G V E V H N A K T K |
| | 931 | CC CG GA GA CA TTCAA AG ACA TAT |
| | | C C A G A C C |
| | | P R E E Q F N S T Y |
| | 961 | AG GT GT TCTGT CT AC GT CTG CAC |
| | | A A A G G C G |
| | | R V V S V L T V L H |
| | 991 | CA GA TG CT AA GG AA GA TAC AAA |
| | | G T G G C C G A |
| | | Q D W L N G K E Y K |
| | 1021 | TGTA GT AG AA AA GG TTACCC AGT |
| | | A G C C G C |
| | | C K V S N K G L P S |
| | 1051 | AG AT GA AA AC ATTAG AA GCA AAA |
| | | C C G G C C A |
| | | S I E K T I S K A K |
| | 1081 | GG CA CC CG GA CC CA GT TAC ACC |
| | | T A C G G C G G |
| | | G Q P R E P Q V Y T |
| | 1111 | CT CCTCC AG CA GA GA AT ACA AAG |
| | | G G C A G A G |
| | | L P P S Q E E M T K |
| | 1141 | AA CA GT AG CT AC TG CT GTG AAG |
| | | T G G C G C C G |
| | | N Q V S L T C L V K |
| | 1171 | GG TTTTA CC AG GA ATTGC GTG GAG |
| | | A C C C T T |
| | | G F Y P S D I A V E |
| | 1201 | TG GA AG AA GG CA CC GA AAT AAC |
| | | G G C T G A A G |
| | | W E S N G Q P E N N |
| | 1231 | TA AA AC AC CC CC GT TTAGAC TCG |
| | | C A G C A A A |
| | | Y K T T P P V L D S |
| | 1261 | GA GG TCTTTCTTCTTGTATTC CGG CTG |
| | | C A G |
| | | D G S F F L Y S R L |
| | 1291 | AC GT GA AA AG AG TG CA GAG GGC |
| | | T G C G C A G A |
| | | T V D K S R W Q E G |
| | 1321 | AA GTTTTCAG TGTAG GTTAT CAC GAA |
| | | T C C G |
| | | N V F S C S V M H E |
| | 1351 | GC CT CA AA CA TATAC CA AAA AGC |
| | | G G C T T T G |
| | | A L H N H Y T Q K S |
| | 1381 | CT AG TTGAG TTAGG AA TG (SEQ ID NO: 21) |
| | | G C C A A A |
| | | L S L S L G K * (SEQ ID NO: 22) |
| Anti-IgE formatted as IgG2 | 1 | ATG GGC TGG TCC TGC ATC ATC CTG TTT CTG |
| variant | | M G W S C I I L F L |
| | 31 | GTG GCC ACA GCC ACC GGC GTG CAC TCT GAA |
| | | V A T A T G V H S E |
| | 61 | GTG CAG CTG GTG GAA TCT GGC GGC GGA CTG |
| | | V Q L V E S G G G L |
| | 91 | GTG CAG CCT GGC GGA TCT CTG AGA CTG AGC |
| | | V Q P G G S L R L S |
| | 121 | TGT GCC GTG TCC GGC TAC AGC ATC ACC AGC |
| | | C A V S G Y S I T S |
| | 151 | GGC TAC TCT TGG AAC TGG ATC CGG CAG GCC |
| | | G Y S W N W I R Q A |
| | 181 | CCT GGC AAG GGA CTG GAA TGG GTG GCC TCC |
| | | P G K G L E W V A S |
| | 211 | ATC ACC TAC GAC GGC AGC ACC AAC TAC AAC |
| | | I T Y D G S T N Y N |
| | 241 | CCC AGC GTG AAG GGC CGG ATC ACC ATC AGC |
| | | P S V K G R I T I S |
| | 271 | AGG GAC GAC AGC AAG AAC ACA TTC TAC CTG |
| | | R D D S K N T F Y L |
| | 301 | CAA ATG AAC AGC CTG CGG GCC GAG GAC ACC |
| | | Q M N S L R A E D T |
| | 331 | GCC GTG TAC TAT TGT GCC AGA GGC AGC CAC |
| | | A V Y Y C A R G S H |
| | 361 | TAC TTC GGC CAC TGG CAC TTT GCC GTG TGG |
| | | Y F G H W H F A V W |
| | 391 | GGC CAG GGA ACA ACG GTC ACC GTG TCT GCA |
| | | G Q G T T V T V S A |
| | 421 | GCG TCG ACA AAA GGC CCT AGC GTC TTT CCA |
| | | A S T K G P S V F P |
| | 451 | CTC GCC CCA TGT TCA AGA AGC ACC AGC GAG |
| | | L A P C S R S T S E |
| | 481 | TCA ACT GCG GCT TTG GGC TGC TTG GTG AAG |
| | | S T A A L G C L V K |
| | 511 | GAT TAC TTC CCA GAG CCT GTC ACT GTC AGC |
| | | D Y F P E P V T V S |
| | 541 | TGG AAT AGC GGG GCT CTG ACC AGT GGA GTG |
| | | W N S G A L T S G V |
| | 571 | CAC ACC TTT CCC GCC GTG TTA CAG AGC AGC |
| | | H T F P A V L Q S S |
| | 601 | GGC CTC TAT AGC CTG AGC AGT GTG GTA ACT |
| | | G L Y S L S S V V T |
| | 631 | GTG CCC TCG AGC AAT TTT GGT ACC CAG ACC |
| | | V P S S N F G T Q T |
| | 661 | TAT ACA TGC AAT GTC GAT CAT AAA CCC AGT |
| | | Y T C N V D H K P S |
| | 691 | AA AC AA GT GA AA AC GT GA AGA |
| | | C A G A C G A C A |
| | | N T K V D K T V E R |
| | 721 | AA TG TGTGT GA TG CCTCCTTG CCC |
| | | A C G G C C |
| | | K C C V E C P P C P |
| | 751 | GC CC CC GT GC GG CCTTC GTTTTT |
| | | C C A G C G G |
| | | A P P V A G P S V F |
| | 781 | CTTTTTCC CCTAA CC AA GA AC CTG |
| | | A A G A C C |
| | | L F P P K P K D T L |
| | 811 | AT AT AG AG AC CC GA GT AC TGT |
| | | G C C G A A A G A |
| | | M I S R T P E V T C |
| | 841 | GT GT GT GA GT TCCCA GA GA CCG |
| | | C C G C A T A C |
| | | V V V D V S H E D P |
| | 871 | GA GT CA TTCAA TG TATGTTGA GGT |
| | | G G A C G T |
| | | E V Q F N W Y V D G |
| | 901 | GT GA GT CA AA GC AA AC AA CCA |
| | | G A G T T A A T G |
| | | V E V H N A K T K P |
| | 931 | CG GA GA CA TTCAA AG AC TTCAGA |
| | | G G A G T C A |
| | | R E E Q F N S T F R |
| | 961 | GT GTTAG GTTCT AC GT GT CA CAA |
| | | A C G T C G C |
| | | V V S V L T V V H Q |
| | 991 | GA TG CT AA GG AA GA TA AA TGT |
| | | T G G C G G A C G |
| | | D W L N G K E Y K C |
| | 1021 | AA GT AG AA AA GG CT CC GC CCA |
| | | G G C C A T G C T |
| | | K V S N K G L P A P |
| | 1051 | ATTGA AA AC ATTTCTAA AC AA GGA |
| | | G A A G C A |
| | | I E K T I S K T K G |
| | 1081 | CA CC CG GA CC CA GT TATAC CTG |
| | | G C G G A G G T |
| | | Q P R E P Q V Y T L |
| | 1111 | CC CCTAG AG GA GA AT AC AA AAC |
| | | G C G G A G C G |
| | | P P S R E E M T K N |
| | 1141 | CA GT AG CT AC TGTCT GT AA GGC |
| | | G G C G C G G A |
| | | Q V S L T C L V K G |
| | 1171 | TTCTA CC AG GA AT GC GTTGA TGG |
| | | C C C T C A G |
| | | F Y P S D I A V E W |
| | 1201 | GA AG AA GG CA CC GA AA AA TAC |
| | | G C T C A C G C C |
| | | E S N G Q P E N N Y |
| | 1231 | AA AC AC CC CC AT CTTGA AG GAC |
| | | G T G C C G T C |
| | | K T T P P M L D S D |
| | 1261 | GG TC TTTTTCCT TA TC AA CT ACC |
| | | A A G C A A G |
| | | G S F F L Y S K L T |
| | 1291 | GT GA AA AG AG TG CA CA GG AAC |
| | | G C A C A G G G A |
| | | V D K S R W Q Q G N |
| | 1321 | GTTTTCAG TG TC GT AT CA GA GCg |
| | | T C A G G C A |
| | | V F S C S V M H E A |
| | 1351 | CT CA AA CA TATAC CA AA AG CTG |
| | | G C T T T G A C |
| | | L H N H Y T Q K S L |
| | 1381 | AG TTGAG TTAGG AA TG (SEQ ID NO: 23) |
| | | C L C L A A A (SEQ ID NO: 24) |
| | | S S G K * |
| Anti-IgE formatted as | 1 | AT GG TG TCCTG AT AT CT TTTCTGL |
| IgG2-FEGG-SELF | | G MC G G W S C C C I C I G L F |
| variant | 31 | GT GC AC GC AC GG GT CA TCTGAA |
| Bold and underlined amino | | G C A C C C G C |
| acid sequences correspond to | | V A T A T G V H S E |
| the variation from the WT | 61 | GT CA CT GT GA TCTGG GG GG CTG |
| sequence, eg the portion of | | G G G G A C C A |
| the lower hinge sequence (aa | | V Q L V E S G G G L AGC S AGC |
| residues 232-237) and | 91 | GT CA CCTGG GG TCTCT AG CT SGCCA |
| mutations S267E and L328F | | GVGQP CGAGS GLARGL TCC |
| as disclosed in Table 1. | 121 | TGTGC GT TCCGG TA AG AT AC |
| | | C CAGVS CGCYCS CI CT |
| | 151 | GG TA TCTTG AA TG AT CG CA |
| | | CGCYS GWCNGWCI GRGQ |
| | 181 | CCTGG AA GG CT GA TG GT GC |
| | | C G A G A G G C |
| | | P G K G L E W V A S |
| | 211 | AT AC TA GA GG AG AC AA TA AAC |
| | | C C C C C C C C C |
| | | I T Y D G S T N Y N |
| | 241 | CC AG GT AA GG CG AT AC AT AGC |
| | | C C G G C G C C C |
| | | P S V K G R I T I S |
| | 271 | AG GA GA AG AA AA AC TTCTA CTG |
| | | G C C C G C A C |
| | | R D D S K N T F Y L |
| | 301 | CA AT AA AG CT CG GC GA GA ACC |
| | | A G C C G G C G C |
| | | Q M N S L R A E D T |
| | 331 | GC GT TA TATTGTGC AG GG AG CAC |
| | | C G C C A C C |
| | | A V Y Y C A R G S H |
| | 361 | TA TTCGG CA TG CA TTTGC GT TGG |
| | | C C C G C C G |
| | | Y F G H W H F A V W |
| | 391 | GG CA GG AC AC GT AC GT TCTGCA |
| | | C G A A G C C G |
| | | G Q G T T V T V S A |
| | 421 | GC TC AC AA GG CCTAG GT TTTCCA |
| | | G G A A C C C |
| | | A S T K G P S V F P |
| | 451 | CTCGC CC TGTTC AG AG AC AG GAG |
| | | C A A A C C C |
| | | L A P C S R S T S E |
| | 481 | TC AC GC GC TTGGG TG TTGGT AAG |
| | | A T G T C C G |
| | | S T A A L G C L V K |
| | 511 | GA TA TTCCC GA CCTGT AC GT AGC |
| | | T C A G C T C |
| | | D Y F P E P V T V S |
| | 541 | TG AA AG GG GC CT AC AG GG GTG |
| | | G T C G T G C T A |
| | | W N S G A L T S G V |
| | 571 | CA AC TTTCC GC GT TTACA AG AGC |
| | | C C C C G G C |
| | | H T F P A V L Q S S |
| | 601 | GG CTCTATAG CT AG AG GT GT ACT |
| | | C C G C T G A |
| | | G L Y S L S S V V T |
| | 631 | GT CC TC AG AA TTTGG AC CA ACC |
| | | G C G C T T C G |
| | | V P S S N F G T Q T |
| | 661 | TATAC TG AA GT GA CA AA CC AGT |
| | | A C T C T T A C |
| | | Y T C N V D H K P S |
| | 691 | AA AC AA GT GA AA AC GT GA AGA |
| | | C A G A C G A C A |
| | | N T K V D K T V E R |
| | 721 | AA TG TGTGT GA TG CCTCCTTG CCC |
| | | A C G G C C |
| | | K C C V E C P P C P |
| | 751 | GC CC GA TTTGA GG GG CCTTC GTT |
| | | C C G G G G G |
| | | A P E F E G G P S V |
| | 781 | TTT CTTTTT CC CCTAA CC AA GA ACC |
| | | A A G A C |
| | | F L F P P K P K D T |
| | 811 | CT AT AT AG AG AC CC GA GT ACA |
| | | G G C C G A A A G |
| | | L M I S R T P E V T |
| | 841 | TGTGT GT GT GA GT GA CA GA GAC |
| | | C C G C A G T A |
| | | C V V V D V E H E D |
| | 871 | CC GA GT CA TTCAA TG TATGTTGAT |
| | | G G G A C G |
| | | P E V Q F N W Y V D |
| | 901 | GG GT GA GT CA AA GC AA AC AAG |
| | | T G A G T T A A T |
| | | G V E V H N A K T K |
| | 931 | CC CG GA GA CA TTCAA AG AC TTC |
| | | A G G A G T C A |
| | | P R E E Q F N S T F |
| | 961 | AG GT GTTAG GTTCT AC GT GT CAC |
| | | A A C G T C G |
| | | R V V S V L T V V H |
| | 991 | CA GA TG CT AA GG AA GA TA AAG |
| | | A T G G C G G A C |
| | | Q D W L N G K E Y |
| | 1021 | TGTAA GT AG AA AA GG TTTCC GCT |
| | | G G C C A T C |
| | | C K V S N K G F P A |
| | 1051 | CC ATTGA AA AC ATTTCTAA AC AAA |
| | | A G A A G C |
| | | P I E K T I S K T K |
| | 1081 | GG CA CC CG GA CC CA GT TATACT |
| | | A G C G G A G G |
| | | G Q P R E P Q V Y T |
| | 1111 | CT CC CCTAG AG GA GA AT AC AAG |
| | | G G C G G A G C |
| | | L P P S R E E M T K |
| | 1141 | AA CA GT AG CT AC TGTCT GT AAA |
| | | C G G C G C G G |
| | | N Q V S L T C L V K |
| | 1171 | GG TTCTA CC AG GA AT GC GTTGAG |
| | | C C C C T C A |
| | | G F Y P S D I A V E |
| | 1201 | TG GA AG AA GG CA CC GA AA AAC |
| | | G G C T C A C G C |
| | | W E S N G Q P E N N |
| | 1231 | TA AA AC AC CC CC AT CTTGA AGC |
| | | C G T G C C G T |
| | | Y K T T P P M L D S |
| | 1261 | GA GG TC TTTTTCCT TA TC AA CTG |
| | | C A A G C A A |
| | | D G S F F L Y S K L |
| | 1291 | AC GT GA AA AG AG TG CA CA GGA |
| | | C G C A C A G G G |
| | | T V D K S R W Q Q G |
| | 1321 | AA GTTTTCAG TG TC GT AT CAC |
| | | CNV F TS CCAS GVG MGAA HE |
| | 1351 | GCgCT CA AA CA TATAC CA AAA |
| | | A GLCHTNTHY TT GQAGCKS |
| | 1381 | CT AG TTGAG TTAGG AA TG (SEQ ID |
| | | G C L C L A A A (SEQ ID NO: 25) |
| | | L S S G K * NO: 26) |
| Anti-IgE formatted as | 1 | AT GG TG TCCTG AT AT CT TTTCTG L |
| IgG2-FLGG-SELF | | GMCGGWS CC CI CI GLF |
| variant | 31 | GT GC AC GC AC GG GT CA TCTGAA |
| Bold and underlined amino | | G C A C C C G C |
| acid sequences correspond to | 61 | GT CA CT GT GA TCTGG GG GG CTG |
| the variation from the WT | | G G G G A C C A |
| sequence, eg the portion of | | V Q L V E S G G G L AGC S AGC |
| the lower hinge sequence (aa | 91 | GT CA CCTGG GG TCTCT AG CT S GCC A |
| residues 232-237) and | | G V G Q P CGAGS GLARGL TCC |
| mutations S267E and L328F | 121 | TGTGC GT TCCGG TA AG AT AC |
| as disclosed in Table 1. | | C CAGVS CGCYCSCI CT |
| | 151 | GG TA TCTTG AA TG AT CG CA |
| | | CGCYS GWCNGWCIGRGQ |
| | 181 | CCTGG AA GG CT GA TG GT GC |
| | | C G A G A G G C |
| | | P G K G L E W V A S |
| | 211 | AT AC TA GA GG AG AC AA TA AAC |
| | | C C C C C C C C C |
| | | I T Y D G S T N Y N |
| | 241 | CC AG GT AA GG CG AT AC AT AGC |
| | | C C G G C G C C C |
| | | P S V K G R I T I S |
| | 271 | AG GA GA AG AA AA AC TTCTA CTG |
| | | G C C C G C A C |
| | | R D D S K N T F Y L |
| | 301 | CA AT AA AG CT CG GC GA GA ACC |
| | | A G C C G G C G C |
| | | Q M N S L R A E D T |
| | 331 | GC GT TA TATTGTGC AG GG AG CAC |
| | | C G C C A C C |
| | | A V Y Y C A R G S H |
| | 361 | TA TTCGG CA TG CA TTTGC GT TGG |
| | | C C C G C C G |
| | | Y F G H W H F A V W |
| | 391 | GG CA GG AC AC GT AC GT TCTGCA |
| | | C G A A G C C G |
| | | G Q G T T V T V S A |
| | 421 | GC TC AC AA GG CCTAG GT TTTCCA |
| | | G G A A C C C |
| | | A S T K G P S V F P |
| | 451 | CTCGC CC TGTTC AG AG AC AG GAG |
| | | C A A A C C C |
| | | L A P C S R S T S E |
| | 481 | TC AC GC GC TTGGG TG TTGGT AAG |
| | | A T G T C C G |
| | | S T A A L G C L V K |
| | 511 | GA TA TTCCC GA CCTGT AC GT AGC |
| | | T C A G C T C |
| | | D Y F P E P V T V S |
| | 541 | TG AA AG GG GC CT AC AG GG GTG |
| | | G T C G T G C T A |
| | | W N S G A L T S G V |
| | 571 | CA AC TTTCC GC GT TTACA AG AGC |
| | | C C C C G G C |
| | | H T F P A V L Q S S |
| | 601 | GG CTCTATAG CT AG AG GT GT ACT |
| | | C C G C T G A |
| | | G L Y S L S S V V T |
| | 631 | GT CC TC AG AA TTTGG AC CA ACC |
| | | G C G C T T C G |
| | | V P S S N F G T Q T |
| | 661 | TATAC TG AA GT GA CA AA CC AGT |
| | | A C T C T T A C |
| | | Y T C N V D H K P S |
| | 691 | AA AC AA GT GA AA AC GT GA AGA |
| | | C A G A C G A C A |
| | | N T K V D K T V E R |
| | 721 | AA TG TGTGT GA TG CCTCCTTG CCC |
| | | A C G G C C |
| | | K C C V E C P P C P |
| | 751 | GC CC GA TTTCT GG GG CCTTC GTT |
| | | C C G G G G G |
| | | A P E F L G G P S V |
| | 781 | TTTCTTTTTCC CCTAA CC AA GA ACC |
| | | A A G A C |
| | | F L F P P K P K D T |
| | 811 | CT AT AT AG AG AC CC GA GT ACA |
| | | G G C C G A A A G |
| | | L M I S R T P E V T |
| | 841 | TGTGT GT GT GA GT GA CA GA GAC |
| | | C C G C A G T A |
| | | C V V V D V E H E D |
| | 871 | CC GA GT CA TTCAA TG TATGTTGAT |
| | | G G G A C G |
| | | P E V Q F N W Y V D |
| | 901 | GG GT GA GT CA AA GC AA AC AAG |
| | | T G A G T T A A T |
| | | G V E V H N A K T K |
| | 931 | CC CG GA GA CA TTCAA AG AC TTC |
| | | A G G A G T C A |
| | | P R E E Q F N S T F |
| | 961 | AG GT GTTAG GTTCT AC GT GT CAC |
| | | A A C G T C G |
| | | R V V S V L T V V H |
| | 991 | CA GA TG CT AA GG AA GA TA AAG |
| | | A T G G C G G A C |
| | | Q D W L N G K E Y K |
| | 1021 | TGTAA GT AG AA AA GG TTTCC GCT |
| | | G G C C A T C |
| | | C K V S N K G F P A |
| | 1051 | CC ATTGA AA AC ATTTCTAA AC AAA |
| | | A G A A G C |
| | | P I E T I S K T K |
| | 1081 | GG CA CC CG GA CC CA GT TATACT |
| | | A G C G G A G G |
| | | G Q P R E P Q V Y T |
| | 1111 | CT CC CCTAG AG GA GA AT AC AAG |
| | | G G C G G A G C |
| | | L P P S R E E M T K |
| | 1141 | AA CA GT AG CT AC TGTCT GT AAA |
| | | C G G C G C G G |
| | | N Q V S L T C L V K |
| | 1171 | GG TTCTA CC AG GA AT GC GTTGAG |
| | | C C C C T C A |
| | | G F Y P S D I A V E |
| | 1201 | TG GA AG AA GG CA CC GA AA AAC |
| | | G G C T C A C G C |
| | | W E S N G Q P E N N |
| | 1231 | TA AA AC AC CC CC AT CTTGA AGC |
| | | C G T G C C G T |
| | | Y K T T P P M L D S |
| | 1261 | GA GG TC TTTTTCCT TA TC AA CTG |
| | | C A A G C A A |
| | | D G S F F L Y S K L |
| | 1291 | ACC GTG GAC AAA AGC AGA TGG CAG CAG GGA |
| | | T V D K S R W Q Q G |
| | 1321 | AAC GTT TTC AGT TGC TCA GTG ATG CAC GAA |
| | | N V F S C S V M H E |
| | 1351 | GCg CTG CAC AAT CAT TAT ACT CAG AAA AGC |
| | | A L H N H Y T Q K S |
| | 1381 | CTG AGC TTG AGC TTA GGA AAA TGA (SEQ ID NO: 27) |
| | | L S L S L G K * (SEQ ID NO: 28) |
| Anti-IgE light chain | 1 | AT GG TG TCCTG AT AT CT TTTCTGL |
| | | GMCGGWS CCCI CI GLF |
| | 31 | GT GC AC GC AC GG GT CA AG GAT |
| | | G C A C C C G C C |
| | | V A T A T G V H S D |
| | 61 | AT CA CT AC CA AG CC AG AG CTG |
| | | C G G A G A C C C |
| | | I Q L T Q R P S S L |
| | 91 | TCTGC AG GT GG GA AG GT AC ATC |
| | | C C G C C A G C |
| | | S A S V G D R V T I |
| | 121 | AC TGTAG GC AG CA AG GT GA TAC |
| | | C A C C G C G C |
| | | T C R A S Q S V D Y |
| | 151 | GA GG GA AG TA AT AA TG TATCAG |
| | | C C C C C G C G |
| | | D G D S Y M N W Y Q |
| | 181 | CA AA CC GG AA GC CC AA CT CTG |
| | | G G C C G C C G G |
| | | Q K P G K A P K L L |
| | 211 | AT TA GC GC AG TA CT GA AG GGC |
| | | C C C C C C G A C |
| | | I Y A A S Y L E S G |
| | 241 | GT CC AG AG TTTTCCGG AG GG TCT |
| | | G C C A C C C |
| | | V P S R F S G S G S |
| | 271 | GG AC GA TTCAC CT AC AT AG TCC |
| | | C C C C G C C C |
| | | G T D F T L T I S S |
| | 301 | CT CA CC GA GA TTCGC AC TA TAC |
| | | G G C G C C C C |
| | | L Q P E D F A T Y Y |
| | 331 | TG CA CA AG CA GA GA CC TA ACC |
| | | C G G C C G C C C |
| | | C Q Q S H E D P Y T |
| | 361 | TTTGG CA GG AC AA GT GA AT AAG |
| | | C G C C G G A C |
| | | F G Q G T K V E I K |
| | 391 | CG AC GT GC GC CC AG GT TTCATC |
| | | G A G C T C C G |
| | | R T V A A P S V F I |
| | 421 | TTCCC CCTAG GA GA CA CT AA TCC |
| | | A C C G G G G |
| | | F P P S D E Q L K S |
| | 451 | GG AC GC TCTGT GT TG CT CT AAC |
| | | C A C C G C G G |
| | | G T A S V V C L L N |
| | 481 | AA TTCTA CC CG GA GC AA GT CAG |
| | | C C C C G C G G |
| | | N F Y P R E A K V Q |
| | 511 | TG AA GT GA AA GC CT CA AG GGC |
| | | G G G C T C G G C |
| | | W K V D N A L Q S G |
| | 541 | AA AG CA GA AG GT AC GA CAG GAC |
| | | C C G A C G C G |
| | | N S Q E S V T E Q D |
| | 571 | AG AA GA TCCAC TA AG CT AGC AGC |
| | | C G C C C C G |
| | | S K D S T Y S L S S |
| | 601 | AC CT AC CT AG AA GC GA TAC GAG |
| | | C G A G C G C C |
| | | T L T L S K A D Y E |
| | 631 | AA CA AA GT TA GC TG GA GTG ACC |
| | | G C G G C C C A |
| | | K H K V Y A C E V T |
| | 661 | CA CA GG CT TCTAG CC GT ACC AAG |
| | | C G C G C C G |
| | | H Q G L S S P V T K |
| | 691 | AG TTCAA CG GG GA TG TA (SEQ ID NO: 29) |
| | | C C G C G C A |
| | | S F N R G E C * (SEQ ID NO: 30) |

Expression and Production of Human IgG and Mutant IgG Proteins by Expi293 Cells

The human IgG and mutant human IgGs were produced in Expi293 human embryonic kidney cells as described previously (Wines et al., 2016 supra). Briefly, Expi293 cells were maintained in Expi293 Expression Medium (Gibco, Waltham, MA, United States of America) for both cell growth and protein production. Cells were transfected simultaneously with the IgG heavy chain plasmid (15 ug) and light chain plasmid (15 ag) diluted in Opti-MEM I Reduced-Serum Medium (Gibco) using the Expifectamine transfection kit (Life Technologies Corporation, Carlsbad, CA, United States of America) then cultured for four days. Culture supernatants were clarified by centrifugation and filtered through a 0.2 μm filter after which the IgGs were purified by affinity chromatography using a Hi-Trap HP Protein A column (GE Healthcare Life Sciences, Marlborough, MA, United States of America) and eluted with 0.1 M citric acid, pH 3.5, followed by neutralisation with 1 M Tris-HCl, pH 9.0. and dialysation against PBS pH 7.5. Any aggregates were removed by subsequent gel filtration on a Superose 6 10/300 GL column (GE Healthcare Biosciences) and monomeric IgG peak fractions collected. The antigen binding activity of all antibody preparations was tested on BSA-TNP by ELISA as described (Wines et al, 2016 supra).

Flow Cytometric Measurement of IgG Binding to Cell Surface FcγR

Antibody FcγR binding was measured using either immune complexes or monomeric IgG. Immune complexes were generated by incubating the anti-TNP antibodies (parental or mutant anti-TNP IgG) with TNP-BSA at a 2:1 ratio (40 μg/ml:20 μg/ml) for 30 minutes at 37° C. then 10 minutes at 4° C. In the flow cytometry binding analysis, the complexes or monomeric IgG, at the indicated concentrations, were added to 25 μl of FcγR-expressing cells (5×106/ml) in PBS/BSA buffer and incubated for one hour on ice, washed twice, resuspended in 50 ul of Alexa 647-conjugated F(ab′)2 fragments of goat anti-human IgG F(ab′)2-specific goat antiserum (Jackson ImmunoResearch Laboratories, West Grove, PA, United States of America) (1/400 dilution in buffer) for 1 hour on ice. The cells were washed twice, resuspended in 200 d PBS/0.5% BSA and 10,000 viable cells analysed in at least three experiments. Monomer IgG binding (MFI) were fitted to a single binding site model to determine binding affinity (KA). Similarly immune complex binding is reported as apparent affinity (K app).

Affinity measurements of IgG:FcR interaction using Bio-Layer Interferometry (OCTET) TNP-BSA was reacted with the EZ-Link™ biotinylation reagent (ThermoFisher Scientific) according to the manufacturer's instructions. The resulting TNP-BSA-biotin (5 μg/ml, 30 sec) was captured to ˜0.8 nm on streptavidin BLI probes using an Octet Red96 (ForteBio; Molecular Devices LLC, San Jose, CA, United States of America) then loaded with anti-TNP IgG (4 μg/ml, 75 sec). A baseline was established (60 sec) followed by the association and dissociation (90s) of a concentration series (15 nM to 20 μM) of rsFcγRIIa-R131 or rsFcγRIIb. Regeneration after each initial reaction TNP-BSA-biotin capture and the subsequent binding cycles used 10 mM HCl. Sensograms were filled to 1:1 Langmuir binding model or the binding response at the end of the association cycle was fitted for steady state affinity.

Production and Purification of Honey Bee Venom Allergen: Api m 1

The major allergen from Honey bee (Apis mellifera) venom: phospholipase A2 (Api m 1) (GenBank X16709, allergen name: Api m 1), was produced in the suspension-adapted insect cell line Spodoptera frugiperda Sf21 as per the manufacturer's instructions (Gibco). Briefly, Sf21 cells were maintained in Sf-900 II SFM media at 27° C. for growth, virus production and protein production. The cDNA encoding full length Api m 1, with a 3′ hexa-His tag, was cloned into the donor plasmid pFastBac that was then transfected into DH10Bac E. coli. The resultant Bacmid DNA was purified from the DH10Bac E. coli cells and transfected into Sf21 cells. The recombinant Baculovirus was then used to infect Sf21 cells and the secreted Api m 1 was purified from cell culture supernatant by Talon Superflow Metal Affinity chromatography (Clontech, Mountain View, CA, United States of America).

Basophil Activation Test (BAT)

BAT assays were performed as previously described (Drew A C et al., J Immunol 173(9):5872-5879, 2004) using either of two cell sources, namely whole (unprocessed) blood or blood washed twice in DMEM/0.1% BSA. Basophils were stimulated using haptenated (TNP) rabbit F(ab′)2 anti-human IgE (anti-IgE-TNP) or haptenated bee venom allergen (Api m 1-TNP) in the presence or absence of the IgG mAbs. Briefly, 100 μl heparinised whole human blood, from either healthy donors or allergic patients was incubated with 20 μl of stimulation buffer (133 mM NaCl, 20 mM Hepes, 7 mM CaCl₂), 5 mM KCl, 3.5 mM MgCl2, 1 mg/ml BSA, 20 μl/ml heparin, 2 ng/ml IL3, pH 7.4) for 10 min at 370° C. Samples were then stimulated, for 20 min at 37° C., by addition of 100 μl of either anti-hIgE-TNP (20 μg/ml) or Api m 1-TNP (4 μg/ml) that had been pre-complexed (37° C. for 30 minutes) with anti-TNP hIgGs. Background stimulation was determined by the addition of 100 μl of stimulation buffer alone. Positive controls for stimulation utilised either N-formyl-Met-Leu-Phe (fMLP, 9 ug/ml) (Sigma-Aldrich) or intact rabbit anti-human IgE (10 μg/ml) (Dako Agilent, Santa Clara, CA, United States of America). The assays were terminated by incubation on ice for 5 min, then normal goat serum added (10 μl)(Sigma-Aldrich). Stimulation responses were quantified by flow cytometry; therefore, following stimulation, the cells were stained for 40 min on ice by the addition of mouse anti-human CD63-PE (2 ul/test) (BD Biosciences, Franklin Lakes, NJ, United States of America), mouse anti-human IgE-FITC (3 ul/test) (eBioscience, San Diego, CA, United States of America) and mouse anti-human CD203c-APC (5 ul/test) (Miltenyi Biotec, Auburn, CA, United States of America). Following staining, red blood cells (RBC) were lysed by incubating twice with 2 ml of lysing solution (154 mM NH4Cl, 10 mM KHCO2, 0.8 mM EDTA) for 10 min at room temperature and centrifugation (250×g, 5 min). Cell pellets were washed with 3 ml wash buffer (133 mM NaCl, 20 mM Hepes, 5 mM KCl, 0.27 mM EDTA, pH 7.3) and resuspended for flow cytometry analysis in 200 μl wash buffer containing 7-Aminoactinomycin D (2 μl/test) (BD Biosciences) for the exclusion of non-viable cells.

Cells were analysed on a FACS CantoII cytometer and fluorescence data analysed using Flowlogic analysis software. The gating strategy used was as follows: Washed blood or whole blood cells were gated on forward and side scatter to include the basophil population, followed by live cell gating based on exclusion of 7AAD positive cells. Basophils were identified as the high IgE expressing (FITC high) and CD203c (APC positive) cells which were then used to set the CD63 negative gate (stimulation buffer alone) and the CD63 positive gate (stimulated with anti-human IgE or fMLP). Activated basophils were identified as the cells in the CD63 positive gate. The % inhibition of basophil activation was calculated as the % reduction in the CD63 positive cells induced by Api m 1-TNP:IgG or anti-IgE-TNP:IgG complexes compared to Api m 1-TNP or anti-IgE-TNP stimulation alone.

Blockade of FcγRIIb Inhibitory Action in the Basophil Activation Test (BAT)

The interaction of FcγRIIb with anti-IgE-TNP:IgG complexes in the BAT was blocked by incubation of cells with F(ab′)2 fragments of the FcγRIIb-specific blocking mAb, H2B6, prior to addition of anti-IgE-TNP:IgG in the BAT as described above. Briefly, 10 μl of anti-H2B6 F(ab′)2 (final concentration of 7.5 μg/ml), or stim buffer alone, was added to 90 μl of washed blood, incubated on ice for 30 mins, then the BAT performed using anti-IgE-TNP or anti-IgE-TNP:IgG (final concentration of 5 μg/ml), and the % basophil activation determined.

Cell surface co-expression of human high affinity FcεRI complex and inhibitory hFcγRIIb Cells expressing the FcεRI complex (FcεRI α, β, γ) with the inhibitory FcγRIIb were generated by the transduction of IIA1.6 cells with a codon-optimised cDNA encoding each FcR or subunit interrupted by an intervening picornavirus ribosomal skipping 2A peptide (Szymczak A L et al., Nat Biotechnol 22(5):589-594, 2004). Sequences were in the order FcεRIα-P2A-FcεRIβ-T2A-FcεRIγ-F2A-FcγRIIb-translation stop. This was synthesised with flanking gateway attB1 and attB2 sites as a sequence verified 2863 bp synthetic DNA (ThermoFisher, GeneArt, Waltham, MA, United States of America). The synthetic DNA was subcloned into the gateway adapted murine leukemia virus expression vector pMXI-neo. Transient transfection of the Phoenix packaging line and infection of the FcR deficient mouse IIA1.6 cell line was performed as previously described (Powell et al., 2006 supra).

IgE Induced Calcium Mobilisation

IIA1.6 cells co-expressing the inhibitory hFcγRIIb and hFcεRI (α, β, γ) complex were sensitised with IgE by overnight incubation with 0.5 μg/ml IgE (JW8/5/13 mouse/human chimeric anti-NP IgE) (Bruggemann M et al., J Exp Med 166:1351-1361, 1987). The following day, cells were stimulated with 20 μg/ml of anti-IgE-T alone or complexed with anti-TNP IgG mAbs (final concentration, 35 μg/ml) and calcium mobilisation determined (Anania J C et al., 2018 supra).

Cell Surface Co-Expression of a Chimeric Anti-NP IgE BCR and Inhibitory hFcγRIIb

Reporter cells expressing a JW8/5/13 mouse/human chimeric anti-NP (4-hydroxy-3-nitrophenylacetyl) IgE BCR comprising mouse V domains (and human cell surface IgE heavy chain including the transmembrane and cytoplasmic domains) and light chain, and co-expressing the inhibitory FcγRIIb were generated by the transduction of IIA1.6 cells with a codon-optimised cDNA encoding a single polyprotein comprising the light chain, IgE heavy chain and FcγRIIb1 separated by the picornavirus ribosomal skipping P2A and F2A peptides (Szymczak A L et al., Nat Biotechnol 22(5):589-594, 2004). Thus, the polyprotein-encoding DNA sequence was configured with the anti-NP IgE light chain cDNA then the P2A peptide, then the IgE heavy chain, then the F2A peptide then the FcγRIIb1, and then the translation Stop. The mouse/human chimeric anti-NP heavy chain sequence comprised an appropriately joined JW8/5/13 anti-NP IgE DNA sequence (Bruggemann M et al., J Exp Med 166:1351-1361, 1987) and sequence accession number X63693.1 (H. sapiens germline alternatively spliced IgE heavy chain DNA). This was synthesised with flanking gateway attB1 and attB2 sites as a sequence verified synthetic DNA (ThermoFisher, GeneArt). The synthetic DNA was subcloned into the Gateway-adapted murine leukaemia virus expression vector pMXI-neo. Transient transfection of the Phoenix packaging line and infection of the FcR deficient mouse IIA1.6 B cell line was performed as previously described (Powell et al., 2006 supra).

IgE B Cell Receptor (BCR)-Induced Calcium Mobilisation

IIA1.6 cells co-expressing the anti-NP surface IgE and the inhibitory hFcγRIIb were loaded with the calcium indicator Fura-2 and incubated with the IgE-specific therapeutic mAb, omalizumab or the mutants; that is, omalizumab formatted as IgG4, as IgG2, IgG2-FEGG, IgG2-FLGG, IgG2-FEGG-S267E-L328F, IgG2-FLGG-S267E-L328F-(1 μg/ml) followed by the NP-related antigen, NIP(22)BSA (bovine serum albumin derivatised with an average of 22, 4-hydroxy-3-iodo-5-nitrophenylacetyl groups per BSA molecule). Calcium mobilisation was determined (Anania J C et al., 2018 supra).

### Results

Mutant IgG2 antibodies with modified Fe generates potent specific basophil inhibitors A series of inhibitory mAbs were developed using IgG2 as a scaffold for sequence elements from IgG4 and IgG1 (Table 1), focussing on the lower hinge region which differs between the IgG subclasses and is a key contact with FcγR. In particular, the VAG of the IgG2 lower hinge was replaced with either FLGG from IgG4 (IgG2-FLGG) or with LLGG from IgG1 (IgG2-LLGG). The mutant IgG2 mAbs were evaluated for their FcγR binding specificity for the different human FcγR expressed on the cell surface. The results are shown in FIG. 1.

The interaction of the mutant IgGs with the human FcγRs on the cell surface was evaluated by flow cytometry and compared to the binding of parental IgG2 or IgG4 as well as to IgG1, the “universal” ligand for all human FcγR. Binding to the low affinity human FcγR (FcγRIIb, FcγRIIa, and FcγRIII) was performed using immune complexes (FIG. 1A-E). Binding to the high affinity FcγRI was determined using monomeric IgG (FIG. 1F). Each of the parental IgG molecules showed the expected specificity (ie complexed IgG1 bound to all receptors, IgG2 failed to bind any FcγR with the exception of FcγRIIa-H131 and IgG4 complexes bound only to the inhibitory FcγRIIb). Uncomplexed, monomeric IgG1 and IgG4, but not IgG2, bound to the high affinity FcγRI.

It was found that the IgG2-LLGG or IgG2-FLGG mAbs FcγR binding profiles were considerably different from IgG2 and in the case of IgG2-FLGG was also distinct from IgG4 (FIG. 1A-E). The IgG2-LLGG mAb showed a specificity profile equivalent to the parental IgG1 (FIG. 1A-E). Thus, the replacement of only the lower hinge in IgG2 was sufficient to confer IgG1-like binding that now included binding to the inhibitory FcγRIIb. Further, the receptor binding profile of IgG2-FLGG was distinct from both parental IgGs (FIG. 1). In particular, this mutant mAb showed significantly enhanced binding to the inhibitory FcγRIIb as well as broader specificity compared to IgG4, binding avidly to both FcγRIIIa allelic forms which do not bind IgG2 or IgG4 and also to the FcγRIIa-R131, also normally a poor binder of IgG2 and IgG4. The binding to FcγRIIa-H131 is presumably a contribution from the IgG2 backbone. However, like monomeric IgG4, monomeric IgG2-FLGG bound to the high affinity FcγRI (FIG. 1F) which does not bind IgG2.

Mutant IgG2 Antibodies Inhibit Api m 1 Allergen-Induced Basophil Activation

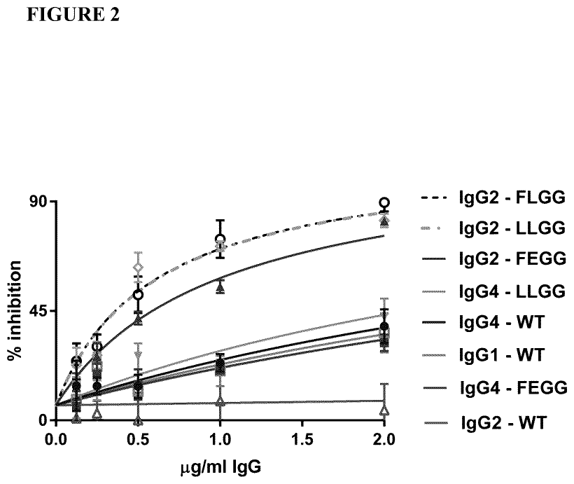

The mutant IgG2 mAbs were also evaluated for their capacity to mediate FcγRIIb-dependent inhibition of allergic basophil activation by IgE. In particular, the IgG2-FLGG, IgG4-LLGG and IgG2-LLGG, antibodies were compared to the parental IgG2, IgG4, and IgG1 for their capacity to modulate FcεRI activation of basophils from IgE+ atopic individuals (honey bee venom allergic patients). The basophils in washed blood were stimulated with the major honey bee venom allergen, phospholipase A2 (Api m 1-TNP) in the presence of the anti-TNP IgG2 or IgG4 mAbs. The results are shown in FIG. 2.

Most strikingly, near-complete inhibition of Api m 1 induced basophil activation was mediated by the IgG2-LLGG and IgG2-FLGG (81% and 85% inhibition respectively), but as expected, the parental IgG2, which does not bind to FcγRIIb, did not inhibit the Api m 1 response. Surprisingly, in contrast, the parental IgG4 and also IgG1, both of which bind avidly to FcγRIIb, showed comparatively weak inhibition achieving only 42% and 45% inhibition respectively at the highest concentration used (2 μg/ml).

Mutation of the Lower Hinge Improves mAb Specificity for FcγRIIb

Specificity for FcγRIIb interaction was further refined by an additional mutation of the lower hinge (FIG. 1) and evaluated for potency in the BAT assay using washed blood (ie plasma-free) or whole blood (ie containing physiological levels of IgG). The results are shown in FIGS. 2, 3 and 7.

First, a point mutation of L235E was introduced into FLGG of the lower hinge sequence of parental IgG4-WT and of IgG2-FLGG mAbs to create IgG4-FEGG and IgG2-FEGG respectively. This mutation has been described as ablating FcγR binding generally (Alegre M L et al., J Immunol 148(11):3461-3468, 1992), and has been used for inactivation of FcγR binding in a number of antibodies (Reddy M P et al., J Immunol 164(4):1925-1933, 2000). However, it was found here that binding to FcγRIIb is retained.

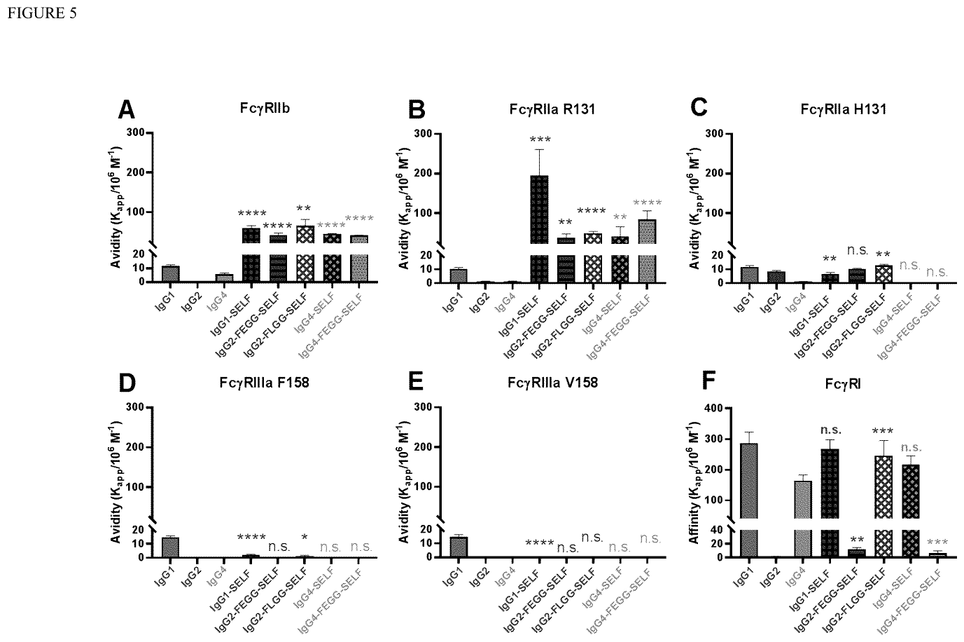

The L235E mutation in IgG2-FEGG and IgG4-FEGG ablated binding to FcγRI of the original unmodified IgG2-FLGG and IgG4-WT down to the near base-line levels of parental IgG2-WT (FIG. 1F). Other backbone-dependent differences were also apparent. On the IgG2 backbone, the IgG2-FEGG and IgG2-FLGG mutants showed similar low affinity FcγR binding profiles (FIG. 1A-E) including the ability to bind readily to FcγRIIb but also to the FcγRIIa-H131 and R131 alleles with only a small comparative decrease in binding of IgG2-FEGG to both alleles of FcγRIIIa (FIG. 1D, E). However, this contrasted starkly with the effect of the same mutation on the IgG4 backbone in the IgG4-FEGG mAb (FIG. 1), and greatly diminished binding in the context of the SELF mutation (FIG. 5F).

The mutants were then evaluated in the washed blood BAT (FIG. 2). Here, and despite an apparent preferential and improved binding to FcγRIIb (see FIG. 1A), IgG4-FEGG showed a surprisingly weak level of inhibition of basophil activation (42%) that was substantially equivalent to that of IgG4-WT (45%). In contrast, the equivalent sequence on the IgG2 backbone, IgG2-FEGG antibody, retained the more potent inhibition seen with the original IgG2-FLGG mutant and also the IgG2-LLGG (FIG. 2).

Modifying CH2 for Improved mAb Affinity and Inhibitory Potency

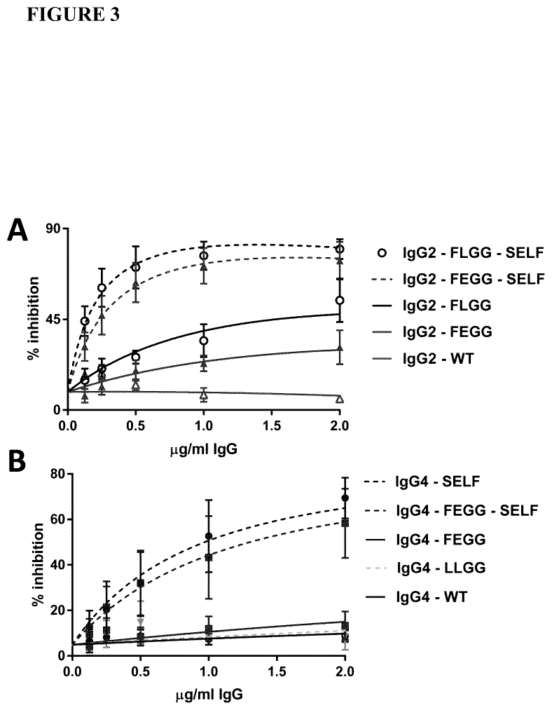

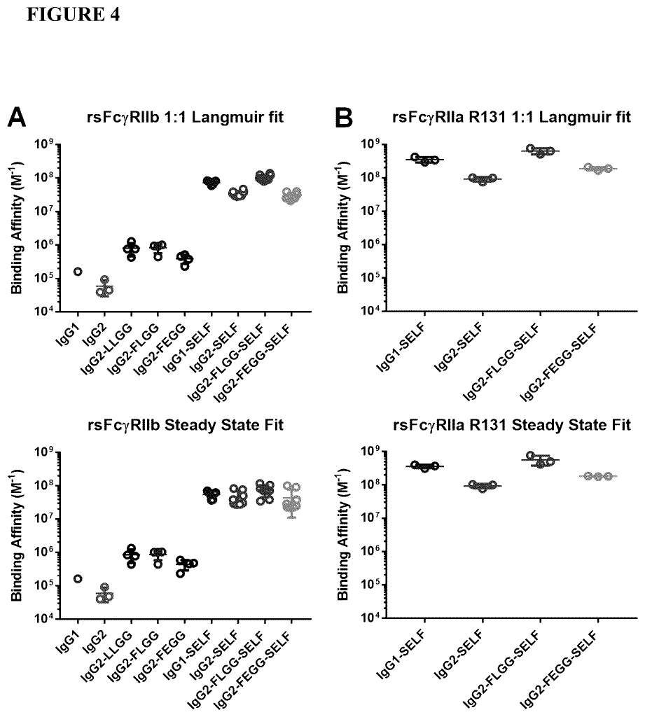

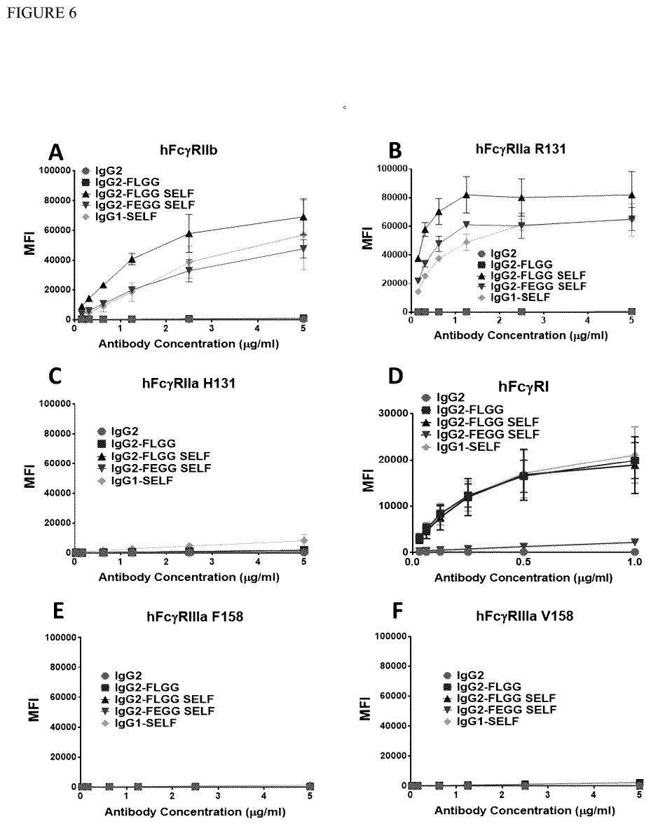

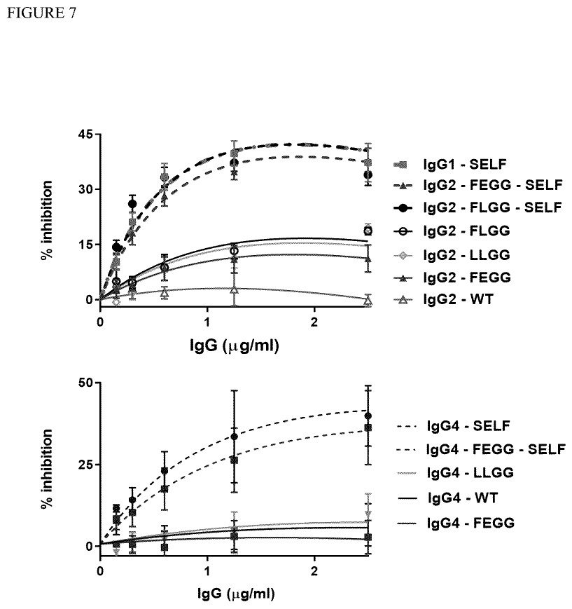

In an attempt to further improve FcγRIIb specificity, two residues in CH2 of the Fc domain were additionally modified (see Table 1). Particularly, two mutations S267E and L328F (SELF) which have been used in the IgG1 backbone (Chu S Y et al., Mol Immunol 45(15):3926-3933, 2008) were introduced into the IgG2- and IgG4-based mAbs, IgG2-FLGG, IgG2-FEGG and IgG4-FEGG, as well as the parental IgG4 antibody. The antibodies were then tested for specificity (see FIGS. 4-6) and inhibitory potency in allergen specific (FIG. 3) and anti-IgE BAT (FIG. 7). Several effects were apparent. That is, the introduction of the SELF mutations resulted in significant increases of affinity (more than 70 fold increases) and altered specificity of binding of both monomeric (FIG. 6) and complexed IgG (FIG. 5). Monomeric IgG2-FLGG-SELF and IgG2-FEGG-SELF showed high affinity binding to FcγRIIb expressed on the cell surface (KA 89 and 32×106 M−1) respectively (FIG. 6A) and by BLI analysis (KA 103 and 29×106 M−1) (FIG. 4 and Table 4), which are 70-120 fold increased affinity compared to the equivalent mAbs without the SELF mutations (FIG. 4 and Table 4). The IgG1-SELF mutant showed high affinity binding as previously reported (Chu S Y et al., Mol Immunol 45:3926-3933, 2008). Notably, these antibodies with the SELF mutations showed (4 to 6-fold) greater binding affinity for FcγRIIa-R131 than for inhibitory FcγRIIb on cells (FIG. 6A, B and confirmed by BLI analysis, as shown in FIG. 4 and Table 4).

This newly acquired high affinity binding was also reflected in a greatly increased binding avidity of immune complexes (FIG. 5). In addition, the unexpected immune complex binding of the original IgG2-FLGG and IgG2-FEGG mAbs to the FcγRIII forms (FIG. 1D, E) was ablated by the SELF mutations (FIG. 5). The effects of inclusion of the SELF-mutations on the IgG2 backbone was also evident on the equivalent IgG4 mAbs (FIG. 5) which showed similarly increased binding to FcγRIIb and FcγRIIa-R131, but not to FcγRIIa-H131.

Despite the major alterations to affinity and specificity of the interaction with low-affinity FcγR induced by SELF-mutations, the interaction with FcγRI was unaffected; the IgG2-FLGG, IgG2-FLGG-SELF showed identical binding as did the IgG1-SELF mutant antibody (FIGS. 6F and 7D). Further, it was found, importantly, that the SELF mutations in IgG2-FEGG-SELF and IgG4-FEGG-SELF did not override the ablation of FcγRI binding by the L235E mutation observed in the original IgG2-FEGG or Ig4-FEGG (FIG. 1F). Thus, this combination of antibody mutations produced a more restricted specificity but also robust binding to the inhibitory FcγRIIb.

TABLE 4
Binding affinities (KA (106 M-1)) of recombinant soluble FcγRIIb
and recombinant soluble FcγRIIa-Arg131 to mutant IgG mAbs

| Fc Receptor | IgG1 | IgG2 | IgG2-LLGG | IgG2-FLGG |
|---|---|---|---|---|
| rsFcγRIIb | 0.16 (1) | 0.059 ± .017 (3) | 0.80 ± .18 (4) | 0.83 ± 0.13 (4) |
| rsFcγRIIa-R131 | ND | ND | ND | ND |

| | | | | IgG2-FLGG- | IgG2-FLGG- |
|---|---|---|---|---|---|
| Fc Receptor | IgG2-FEGG | IgG1-SELF | IgG2-SELF | SELF | SELF |
| rsFcγRIIb | 0.40 ± .06 (4) | 74 ± 4 (7) | 34 ± 2 (8) | 103 ± 8 (8) | 29 ± 3 (8) |
| rsFcγRIIa-R131 | ND | 349 ± 38 (3) | 91 ± 8 (3) | 630 ± 75 (3) | 188 ± 12 (3) |

Affinities are from global fitting to Langmuir 1:1 binding model, KA ± S.E.M. (n), n = number of experiments

Inhibition of basophil activation in whole blood from allergic patients and healthy donors The mutant IgG2 mAbs were also evaluated for their inhibitory potency in whole blood; that is, in the presence of physiological levels of IgG using two separate IgE-dependent stimuli, either allergen Api m 1-T (FIG. 3) or anti-IgE-TNP (FIG. 7). In comparison with corresponding IgG4 mutants, the IgG2-based mAbs (FIG. 7A) were more potent inhibitors of allergen induced activation than the IgG4-mAbs (FIG. 7B). Indeed, IgG4, IgG4-FEGG, and IgG4-LLGG which inhibited activation in washed blood (FIG. 2) showed very poor inhibition in whole blood (8%, 8%, 13% inhibition respectively). In contrast, and despite the presence of physiological IgG, the IgG2-FLGG, containing the lower hinge of IgG4 and which binds FcγRIIb with low affinity, retained substantial inhibition (54%, IC50=1.6 μg/ml). In addition, the results showed that the potency of the IgG2 mAbs was greatly increased by the inclusion of the SELF mutations of the CH2 region. For instance, binding of the IgG2-FLGG-SELF and the IgG2-FEGG-SELF was improved at least 6-fold to IC50=0.24 μg/ml and 0.38 μg/ml respectively.

Interestingly, the mutant IgG2 mAbs with SELF mutations were substantially more potent than their IgG4 lower hinge equivalents (eg IgG2-FLGG-SELF having the IgG4 lower hinge (IC50=0.24 μg/ml) compared to IgG4-SELF IC50=1.1 μg/ml, indicating the nature of the IgG backbone is a significant factor in determining inhibitory potency in these engineered antibodies).

The mutant IgG2 mAbs were also tested in a second IgE/FcεRI-dependent system to ensure that the potency was not unique to the allergen system. That is, assays were conducted for basophils in whole blood stimulated with anti-IgE-TNP, and the results showed that the relative potency of the inhibition mediated by the antibodies was the same as that seen using Api m 1-T allergen stimulation; that is, IgG2-FLGG-SELF≈IgG2-FEGG-SELF≈IgG1-SELF>IgG2-FLGG≈IgG2-FEGG>>>IgG2 and for the IgG4 backbone mAbs, IgG4-SELF≈IgG4-FEGG-SELF>>>IgG4-FEGG≈IgG4-LLGG≈IgG4. Thus, the ability of a particular mutant mAb containing to inhibit activation of basophils and its ranking compared to the other antibodies used, was equivalent whether the basophils were activated via anti-hIgE-TNP fragments or Api m 1-TNP.

The Inhibitory Function of the Mutant IgG2 Antibodies is Impaired by FcγRIIb Blockade

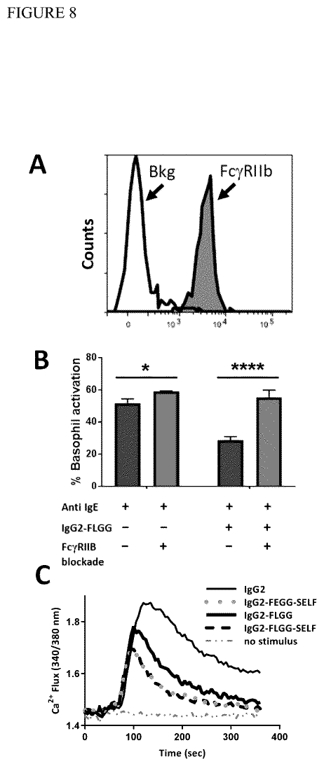

The expected expression of the inhibitory FcγRIIb receptor on blood basophils (Kepley C L et al., J Allergy Clin Immunol 106:337-348, 2000) was confirmed by flow cytometry (see FIG. 8A). The FcγRIIb dependence of the inhibition by the IgG mAbs of IgE/FcεRI basophil activation, was evaluated by the blockade of FcγRIIb expressed on basophils using the FcγRIIb-specific mAb, H2B6 (FIG. 8B). Pre-treatment of whole blood with H2B6 F(ab′)2 fragments prior to stimulation with anti-IgE-TNP, in the presence of IgG2-FLGG, resulted in significant reduction in the potency of IgG2-FLGG (FIG. 8B).

Inhibition of FcεRI Induced Calcium Mobilisation

The mutant IgG2 antibodies were also tested for FcγRIIb-dependent modulation of IgE:FcεRI induced calcium mobilisation (FIG. 8C). Cells co-expressing FcγRIIb and the FcεRI (αβγ2) complex were sensitised with IgE and stimulated with anti-IgE-TNP in the presence of the parental anti-TNP IgG2 or the IgG2-based anti-TNP mutant mAbs (FIG. 8C). The inhibition of the IgE/FcεRI calcium mobilisation by the mAbs correlated with their affinity for FcγRIIb and with their potency of inhibition in both allergen or anti-IgE induced activation of basophils (FIGS. 3 and 8). The IgG2-FLGG-SELF and IgG2-FEGG-SELF antibodies showed the greatest inhibition of calcium mobilisation and produced similar reductions in the magnitude and kinetics of the response. Interestingly, IgG2-FLGG antibody which did not contain the SELF mutations, still showed a substantial reduction in the IgE/FcεRI Ca2+ response (FIG. 8C), which is also consistent with its inhibition of basophil activation in whole blood (FIG. 3).

Inhibition of Surface IgE, B Cell Antigen Receptor-Induced Calcium Mobilisation

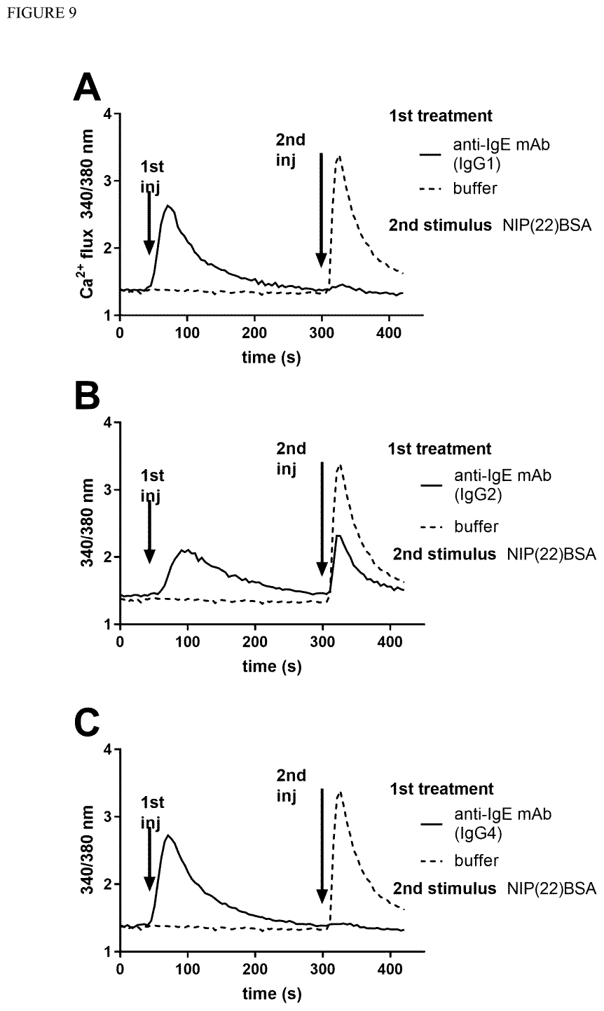

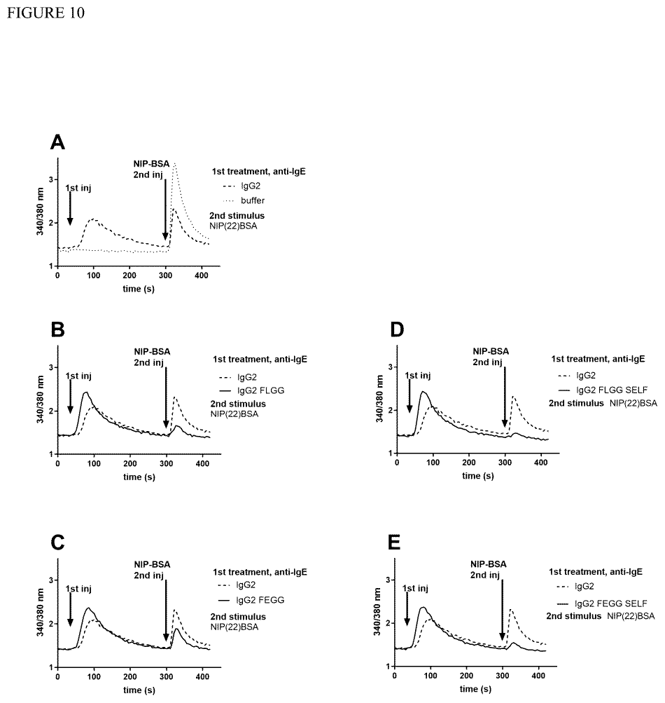

The therapeutic mAb omalizumab was reformatted as an IgG4 and IgG2 antibodies and these were tested for FcγRIIb-dependent modulation of antigen (NIP22BSA) induced calcium mobilisation (FIG. 9 and FIG. 10).

IIA1.6 B cells co-expressing FcγRIIb and NP-specific cell surface IgE BCR were treated with the therapeutic mAb, omalizumab (an IgG1 mAb) or omalizumab mutant mAbs (provided with an IgG4 backbone or as a mutant IgG2 antibody according to the present disclosure) and the BCR subsequently stimulated with the NP-related antigen NIP(22)BSA antigen. The IgG1 omalizumab treatment strongly suppressed the subsequent calcium mobilisation by antigen (second injection, NIP(22)BSA, FIG. 9A). The IgG2-mutant anti-IgE treatment only partially suppressed the subsequent calcium mobilisation by NIP(22)BSA antigen (second injection, FIG. 9B). Like omalizumab, but unlike the IgG2 counterpart, the IgG4-formatted anti-IgE strongly suppressed antigen stimulated calcium mobilisation (FIG. 9C). These suppressive activities correlate with the ability of IgG1 and IgG4 to engage inhibitory FcγRIIb1, while IgG2 is unable to bind FcγRIIb1.

The effect of mAbs targeting the BCR on antigen stimulation was evaluated on IIA1.6 B cells co-expressing FcγRIIb1 using anti-IgE mAbs comprising the variable domains of omalizumab provided with an IgG2 backbone (FIG. 10). Treatment of the B cells co-expressing IgE BCR and the human FcγRIIb1 with IgG2 form of omalizumab resulted in partial suppression of the antigen (NIP)-specific hu-IgE BCR-triggered calcium flux in comparison to the buffer control (second injection NIP(22)BSA, FIG. 10A). This partial suppression is independent of FcγRIIb1 since IgG2 does not bind FcγRIIb.

However, treatment with omalizumab provided as an IgG2-FLGG antibody largely suppressed the antigen-specific hu-IgE BCR triggered calcium flux (second injection, FIG. 10B). Also, omalizumab provided as an IgG2-FEGG mAb also suppressed antigen triggered calcium flux (second injection, FIG. 10C), but less potently than the IgG2-FLGG formatted mAb. The form of omalizumab provided as IgG2-FLGG-SELF mAb also suppressed the antigen-stimulated response (second injection, FIG. 10D) while the suppression by the IgG2-FEGG-SELF form (FIG. 10E) was equivalent to that of the IgG-FLGG mAb. Overall, regulation of the IgE BCR had the hierarchy of FLGG-SELF>FLGG˜FEGG-SELF>FEGG>IgG2, which broadly correlated with the rank order of FcγRIIb binding activity of these mutations in this IgG2 format.

### DISCUSSION

The targeting of immune checkpoints has emerged as a significant strategy for modulating leukocyte responses in disease. FcγRIIb is one of the earliest immune checkpoints described (Hibbs M L et al., Proc Natl Acad Sci USA 83:6980-6984, 1986). Its modulation of ITAM-dependent signalling pathways utilised by FcεRI, other activating type FcRs and the B cell antigen receptor, regulates antibody-dependent leukocyte function in innate and adaptive immunity. This includes the inhibition of IgE-dependent basophil activation (Cady C T et al., Immunol Lett 130(1-2):57-65, 2010) and the B cell antigen receptor (Amigorena S et al., Science 256:1808-1812, 1992). In the work described in this example, it was found that “functionally inert” human IgG2 can be used as a scaffold for the development of mAbs with modified FcγR specificity/affinity to harness the inhibitory potency of FcγRIIb by mutating the sequence of the lower hinge. Indeed, in this way, it was found to be possible to exploit the inhibitory potency of FcγRIIb so as to modulate the activating-type receptor, FcεRI, to thereby inhibit IgE/FcαRI allergen or anti-IgE activation of human basophils. Moreover, providing an anti-IgE mAb as such mutant IgG2 antibodies was effective for the inhibition of antigen stimulation of a surface IgE B cell receptor. Such mutant IgG2 antibodies therefore offer considerable promise as the basis of novel mAb therapeutics for treating or preventing allergic responses.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the methods and uses of the immunotherapeutic protein (eg mutant IgG2 antibody) and composition disclosed herein are not restricted by the particular application(s) described. Neither are the methods, uses and composition restricted in their preferred embodiment(s) with regard to the particular elements and/or features described or depicted herein. It will also be appreciated that the methods and uses of the immunotherapeutic protein and composition disclosed herein are not limited to the embodiment or embodiments disclosed, but are capable of numerous rearrangements, modifications and substitutions without departing from the scope of the disclosure as set forth and defined by the following claims.

1. A method of treating a disease or condition in a subject, wherein binding to and/or activation of FcγRIIb is beneficial in the treatment or prevention of the disease or condition, the method comprising administering to the subject an effective amount of an immunotherapeutic protein comprising at least one heavy chain polypeptide derived from an IgG2 antibody, wherein the heavy chain polypeptide comprises at least constant heavy domains 2 and 3 (CH2 and CH3) and the lower hinge, and the sequence of the lower hinge comprises a mutation enabling the immunotherapeutic protein to bind to and/or activate FcγRIIb.

2. The method of claim 1, wherein the heavy chain polypeptide is a heavy chain component of an Fc fragment.

3. The method of claim 1, wherein the immunotherapeutic protein is a mutant IgG2 antibody.

4. The method of claim 1, wherein the mutation comprises the substitution of the lower hinge sequence, or the substitution of one or more amino acid(s) within the lower hinge sequence, at positions 233-236 (EU numbering).

5. The method of claim 1, wherein the lower hinge sequence comprises the amino acid sequence:
X¹X²X³-G-X⁵ or
wherein
X¹ is selected from proline (P) and glutamic acid (E),
X² is selected from valine (V), leucine (L) and phenylalanine (F),
X³ is selected from leucine (L), alanine (A) and glutamic acid (E), and
X⁵ is selected from glycine (G) and proline (P), or is absent,
but with the proviso that the lower hinge does not consist of a wild type IgG2 lower hinge sequence.

6. The method of claim 1, wherein the lower hinge sequence comprises an amino acid sequence selected from the group consisting of: ELLGG, EFLGG, EFLGP and EFEGG.

7. The method of claim 1, wherein the immunotherapeutic protein binds to and activates FcγRIIb to recruit FcγRIIb inhibitory function.

8. The method of claim 1, wherein the immunotherapeutic protein binds to FcγRIIb to induce FcγRIIb-mediated endocytosis/internalisation (“sweeping”).

9. The method of claim 1, wherein the immunotherapeutic protein binds to FcγRIIb to induce FcγRIIb-mediated scaffolding.

10. The method of claim 1, wherein the immunotherapeutic protein further comprises S267E and/or L328F amino acid substitution(s) (EU numbering) in the CH2 region of the at least one of the heavy chain polypeptide.

11. The method of claim 1, wherein the lower hinge sequence comprises the amino acid sequence EFLGG.

12. The method of claim 1, wherein the lower hinge sequence comprises the amino acid sequence EFEGG and the immunotherapeutic protein further comprises S267E and/or L328F amino acid substitution(s) (EU numbering) in the CH2 region of the at least one heavy chain polypeptide.

13. The method of claim 1, wherein the immunotherapeutic protein includes no further mutation(s) within the constant heavy region of the heavy chain polypeptides.

14. The method of claim 10, wherein the subject is homozygous for FcγRIIa-H¹³¹.

15. The method of claim 1, wherein the immunotherapeutic protein is a human or humanised monoclonal antibody (mAb).

16. The method of claim 1, wherein the disease or condition to be treated is selected from allergic diseases, autoimmune diseases and conditions, other inflammatory diseases, infectious diseases and proliferative diseases.

17. The method of claim 16, wherein the disease or condition to be treated is an allergic disease and the immunotherapeutic protein comprises an antigen binding region which specifically binds to an allergen.

18. The method of claim 17, wherein the immunotherapeutic protein mediates FcγRIIb-dependent inhibition of allergic basophil activation by IgE.

19. The method of claim 17, wherein the disease or condition to be treated is an autoimmune disease and the immunotherapeutic protein comprises an antigen binding region which specifically binds to an autoantigen.

20. The method of claim 19, wherein the autoimmune disease is systemic lupus erythematosus (SLE) or multiple sclerosis (MS).

21. The method of claim 1, wherein the disease or condition to be treated is a cancer and the immunotherapeutic protein comprises an antigen binding region which specifically binds to a cancer antigen.

22. The method of claim 1, wherein the immunotherapeutic protein comprises an antigen binding region which specifically binds to:
(a) an antigen;
(b) an antibody bound to an activating receptor;
(c) an antibody (ligand) binding domain of an activating receptor;
(d) a subunit of an activating receptor;
(e) an antigen bound to an immunoglobulin component of a BCR complex; or
(f) a subunit of a BCR complex or an associated Ig-α or β chains.

23. A pharmaceutical composition comprising an immunotherapeutic protein as defined in claim 1, and a pharmaceutically acceptable carrier, diluent and/or excipient.


SEQUENCE LISTING (31 sequences)

SEQ ID NO: 1 - protein, 5 aa, Homo sapiens
ELLGG

SEQ ID NO: 2 - protein, 5 aa, synthetic construct
  REGION 1..5: note = Mutant lower hinge sequence derived from human IgG4
EFLGG

SEQ ID NO: 3 - protein, 5 aa, synthetic construct
  REGION 1..5: note = Mutant lower hinge sequence
EFEGG

SEQ ID NO: 4 - skipped

SEQ ID NO: 5 - protein, 5 aa, synthetic construct
  REGION 1..5: note = Lower hinge sequence
EFLGP

SEQ ID NO: 6 - protein, 4 aa, synthetic construct
  REGION 1..4: note = Mutant lower hinge sequence
FLGG

SEQ ID NO: 7 - protein, 4 aa, synthetic construct
  REGION 1..4: note = Mutant lower hinge sequence
LLGG

SEQ ID NO: 8 - protein, 4 aa, synthetic construct
  REGION 1..4: note = Mutant lower hinge sequence
FEGG

SEQ ID NO: 9 - protein, 4 aa, Homo sapiens
CPPC

SEQ ID NO: 10 - protein, 6 aa, Homo sapiens
PELLGG

SEQ ID NO: 11 - protein, 5 aa, Homo sapiens
PPVAG

SEQ ID NO: 12 - protein, 6 aa, synthetic construct
  REGION 1..6: note = FEGG lower hinge sequence
PEFEGG

SEQ ID NO: 13 - protein, 6 aa, Homo sapiens
PEFLGG

SEQ ID NO: 14 - protein, 4 aa, Homo sapiens
PVAG

SEQ ID NO: 15 - DNA, 1398 bp, synthetic construct
  misc_feature 1..1398: note = Anti-TNP mAb formatted as IgG2-FEGG-SELF variant
  source 1..1398: mol_type = other DNA
aagcttaccatggtgcttagtttgctgtatctgctcaccgccctgcctggaatcctgagcgaggtgcagttgcaggaatctggtcccagtctagttaagcccagccagacactgagcctgacatgtagtgtgaccggtgacagcattacaagcggctactggaactggatcagacaggtgccaggaaacaaactcgaatacatggggttcatcaattacagtggcaatacttactataatcccagcctgagaagcagaatttctataaccagagacaccagcaaaaaccagtactttctgcacctgaacagcgtaacaacggaagacaccgccacctattactgcgccagggccaactgggatgtgttcgcatactggggccagggcactctggtgacggtgagcgccgcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccgagtttgagggggggccttcggtttttctttttccacctaaaccgaaagacaccctgatgatcagcaggacaccagaagtgacatgtgtcgtcgtggacgtagagcatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggttttcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaatgatctaga

SEQ ID NO: 16 - protein, 460 aa, synthetic construct
  REGION 1..460: note = Anti-TNP mAb formatted as IgG2-FEGG-SELF variant
MVLSLLYLLTALPGILSEVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFEGGPSVFLFPPKPKDTLMISRTPEVTCVVVDVEHEDPEVQFNWYVDGVEVHNAKTKPREEQFNSTFRVVSVLTVVHQDWLNGKEYKCKVSNKGFPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 17 - DNA, 1398 bp, synthetic construct
  misc_feature 1..1398: note = Anti-TNP mAb formatted as IgG2-FLGG-SELF variant
  source 1..1398: mol_type = other DNA
aagcttaccatggtgcttagtttgctgtatctgctcaccgccctgcctggaatcctgagcgaggtgcagttgcaggaatctggtcccagtctagttaagcccagccagacactgagcctgacatgtagtgtgaccggtgacagcattacaagcggctactggaactggatcagacaggtgccaggaaacaaactcgaatacatggggttcatcaattacagtggcaatacttactataatcccagcctgagaagcagaatttctataaccagagacaccagcaaaaaccagtactttctgcacctgaacagcgtaacaacggaagacaccgccacctattactgcgccagggccaactgggatgtgttcgcatactggggccagggcactctggtgacggtgagcgccgcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccgagtttctgggggggccttcggtttttctttttccacctaaaccgaaagacaccctgatgatcagcaggacaccagaagtgacatgtgtcgtcgtggacgtagagcatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggttttcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaatgatctaga

SEQ ID NO: 18 - protein, 463 aa, synthetic construct
  REGION 1..463: note = Anti-TNP mAb formatted as IgG2-FLGG-SELF variant
KLTMVLSLLYLLTALPGILSEVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVEHEDPEVQFNWYVDGVEVHNAKTKPREEQFNSTFRVVSVLTVVHQDWLNGKEYKCKVSNKGFPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 19 - DNA, 723 bp, synthetic construct
  misc_feature 1..723: note = Chimeric anti-TNP light chain
  source 1..723: mol_type = other DNA
atggattcccaggcccaggtgctcatgctgttactcctgtgggtctccggaacctgcggcgacatagtgatgtcacagagcccaagcagccttgcagtgtctgttggtgagaaggtgaccatgagctgcaagtcaagccagagcctgctctatagcagcaatcagaagaactatttggcctggtatcagagaaaacccggccagagccctaagctccttatttattgggctagcacacgcgagtccggcgtgccggacagatttaccggcagcggtagcggcaccgattttactttgaccatctcgtctgtaaaggccgaagacctggcagtgtattactgtcaacattattactcatctccctacactttcggaggggggaccaaactggaaattaagcgtacggttgctgccccttctgtcttcatcttccctcccagcgatgaacagctgaaaagtgggacagcgagtgtagtgtgcctgctaaacaatttttacccacgggaggccaaagtgcagtggaaagtggacaacgctctgcaaagtggaaattctcaggagtccgtcacagagcaggactcgaaggacagcacttacagcttgtcaagcaccctgacgctgagcaaagccgattacgagaagcacaaggtgtacgcatgcgaagttacgcaccaaggactgagtagcccagtcacaaagagcttcaacaggggggaatgttag

SEQ ID NO: 20 - protein, 240 aa, synthetic construct
  REGION 1..240: note = Chimeric anti-TNP light chain
MDSQAQVLMLLLLWVSGTCGDIVMSQSPSSLAVSVGEKVTMSCKSSQSLLYSSNQKNYLAWYQRKPGQSPKLLIYWASTRESGVPDRFTGSGSGTDFTLTISSVKAEDLAVYYCQHYYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1404 bp, synthetic construct
  misc_feature 1..1404: note = Anti-IgE formatted as IgG4
  source 1..1404: mol_type = other DNA
atgggctggtcctgcatcatcctgtttctggtggccacagccaccggcgtgcactctgaagtgcagctggtggaatctggcggcggactggtgcagcctggcggatctctgagactgagctgtgccgtgtccggctacagcatcaccagcggctactcttggaactggatccggcaggcccctggcaagggactggaatgggtggcctccatcacctacgacggcagcaccaactacaaccccagcgtgaagggccggatcaccatcagcagggacgacagcaagaacacattctacctgcaaatgaacagcctgcgggccgaggacaccgccgtgtactattgtgccagaggcagccactacttcggccactggcactttgccgtgtggggccagggaacaacggtcaccgtgtctgcagcgtcgacaaaaggtcccagcgtgtttcccctggctccttgtagcagaagcacgtcagaaagtacagctgccctgggatgcctggtgaaagattattttcccgagcccgttaccgtttcctggaatagtggcgccctgacctcgggcgtgcatacttttcccgctgttctgcagagcagcggcctgtatagcctgagcagtgtggtgactgttccgtctagcagcctgggtaccaagacttatacatgcaacgtggaccataagcctagcaatactaaggtagataagcgagtggagagcaagtacggccccccctgccctagctgtcccgcacctgagtttctaggcgggcccagcgtgtttctgttccccccgaagcctaaagataccctgatgatcagcagaacacccgaagtgacctgcgtggttgtggacgtcagccaggaagatcctgaggtgcagttcaattggtacgtggacggcgtggaggtgcacaatgctaagacaaagccccgcgaagagcaattcaacagcacatatagagtagtatctgtgctgaccgtgctgcaccaggattggctgaacggcaaggaatacaaatgtaaagtgagcaacaagggcttacccagtagcatcgagaagaccattagcaaagcaaaaggtcaaccccgggagccccaggtgtacaccctgcctccgagccaagaggaaatgacaaagaatcaggtgagcctgacctgcctggtgaagggattttaccccagcgatattgctgtggagtgggagagcaatgggcaaccagagaataactacaaaacgaccccaccagtattagactcggacggatctttcttcttgtattcgcggctgactgtggacaagagcagatggcaagagggcaatgttttcagctgtagcgttatgcacgaagcgctgcacaatcattatactcagaaaagcctgagcttgagcttaggaaaatga

SEQ ID NO: 22 - protein, 467 aa, synthetic construct
  REGION 1..467: note = Anti-IgE formatted as IgG4
MGWSCIILFLVATATGVHSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - DNA, 1401 bp, synthetic construct
  misc_feature 1..1401: note = Anti-IgE formatted as IgG2 variant
  source 1..1401: mol_type = other DNA
atgggctggtcctgcatcatcctgtttctggtggccacagccaccggcgtgcactctgaagtgcagctggtggaatctggcggcggactggtgcagcctggcggatctctgagactgagctgtgccgtgtccggctacagcatcaccagcggctactcttggaactggatccggcaggcccctggcaagggactggaatgggtggcctccatcacctacgacggcagcaccaactacaaccccagcgtgaagggccggatcaccatcagcagggacgacagcaagaacacattctacctgcaaatgaacagcctgcgggccgaggacaccgccgtgtactattgtgccagaggcagccactacttcggccactggcactttgccgtgtggggccagggaacaacggtcaccgtgtctgcagcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaatga

SEQ ID NO: 24 - protein, 466 aa, synthetic construct
  REGION 1..466: note = Anti-IgE formatted as IgG2 variant
MGWSCIILFLVATATGVHSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 25 - DNA, 1404 bp, synthetic construct
  misc_feature 1..1404: note = Anti-IgE formatted as IgG2-FEGG-SELF variant
  source 1..1404: mol_type = other DNA
atgggctggtcctgcatcatcctgtttctggtggccacagccaccggcgtgcactctgaagtgcagctggtggaatctggcggcggactggtgcagcctggcggatctctgagactgagctgtgccgtgtccggctacagcatcaccagcggctactcttggaactggatccggcaggcccctggcaagggactggaatgggtggcctccatcacctacgacggcagcaccaactacaaccccagcgtgaagggccggatcaccatcagcagggacgacagcaagaacacattctacctgcaaatgaacagcctgcgggccgaggacaccgccgtgtactattgtgccagaggcagccactacttcggccactggcactttgccgtgtggggccagggaacaacggtcaccgtgtctgcagcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccgagtttgagggggggccttcggtttttctttttccacctaaaccgaaagacaccctgatgatcagcaggacaccagaagtgacatgtgtcgtcgtggacgtagagcatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggttttcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaatga

SEQ ID NO: 26 - protein, 467 aa, synthetic construct
  REGION 1..467: note = Anti-IgE formatted as IgG2-FEGG-SELF variant
MGWSCIILFLVATATGVHSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFEGGPSVFLFPPKPKDTLMISRTPEVTCVVVDVEHEDPEVQFNWYVDGVEVHNAKTKPREEQFNSTFRVVSVLTVVHQDWLNGKEYKCKVSNKGFPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 27 - DNA, 1404 bp, synthetic construct
  misc_feature 1..1404: note = Anti-IgE formatted as IgG2-FLGG-SELF variant
  source 1..1404: mol_type = other DNA
atgggctggtcctgcatcatcctgtttctggtggccacagccaccggcgtgcactctgaagtgcagctggtggaatctggcggcggactggtgcagcctggcggatctctgagactgagctgtgccgtgtccggctacagcatcaccagcggctactcttggaactggatccggcaggcccctggcaagggactggaatgggtggcctccatcacctacgacggcagcaccaactacaaccccagcgtgaagggccggatcaccatcagcagggacgacagcaagaacacattctacctgcaaatgaacagcctgcgggccgaggacaccgccgtgtactattgtgccagaggcagccactacttcggccactggcactttgccgtgtggggccagggaacaacggtcaccgtgtctgcagcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccgagtttctgggggggccttcggtttttctttttccacctaaaccgaaagacaccctgatgatcagcaggacaccagaagtgacatgtgtcgtcgtggacgtagagcatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggttttcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaatga

SEQ ID NO: 28 - protein, 467 aa, synthetic construct
  REGION 1..467: note = Anti-IgE formatted as IgG2-FLGG-SELF variant
MGWSCIILFLVATATGVHSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVEHEDPEVQFNWYVDGVEVHNAKTKPREEQFNSTFRVVSVLTVVHQDWLNGKEYKCKVSNKGFPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 29 - DNA, 714 bp, Homo sapiens
  source 1..714: mol_type = genomic DNA
atgggctggtcctgcatcatcctgtttctggtggccacagccaccggcgtgcacagcgatatccagctgacacagagacccagcagcctgtctgccagcgtgggcgacagagtgaccatcacctgtagagccagccagagcgtggactacgacggcgacagctacatgaactggtatcagcagaagcccggcaaggcccccaagctgctgatctacgccgccagctacctggaaagcggcgtgcccagcagattttccggcagcggctctggcaccgacttcaccctgaccatcagctccctgcagcccgaggacttcgccacctactactgccagcagagccacgaggacccctacacctttggccagggcaccaaggtggaaatcaagcggacagtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgctaa

SEQ ID NO: 30 - protein, 237 aa, Homo sapiens
MGWSCIILFLVATATGVHSDIQLTQRPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - skipped